(19) United States
(12) Patent Application Publication
(10) Pub. No.: US 2010/0144838 A1
(43) Pub. Date: Jun. 10, 2010

(54) Methods for Identifying Modulators of Pyrimidine Tract Binding Protein

(76) Inventors: William T. Beck, Chicago, IL (US); Xiaolong He, Palatine, IL (US)

Correspondence Address: POLSINELLI SHUGHART PC, 700 W. 47TH STREET, SUITE 1000, KANSAS CITY, MO, 64112-1802, US

(21) Appl. No.: 12/628,808

(22) Filed: Dec. 1, 2009

Related U.S. Application Data
(60) Provisional application No. 61/118,845, filed on Dec. 1, 2008.

(51) Int. Cl. A61K 31/7088; A61P 35/00; G01N 33/53; C12Q 1/68

(52) U.S. Cl. 514/44 A; 435/7.21; 435/6

(57) ABSTRACT

Provided herein are methods and materials for identifying compounds that modulate polypyrimidine tract binding protein (PTB), a protein that functions as a negative regulator of pre-mRNA splicing by blocking the inclusion of numerous alternative exons into mRNA.

### CROSS-RELATED APPLICATIONS

The present application claims the benefit of the filing date of provisional application 61/118,845, filed on Dec. 1, 2008, which is incorporated by reference in its entirety.

### FIELD OF THE INVENTION

The present invention relates to using alternative splicing mechanisms to identify compounds that modulate the activity or expression of polypyrimidine-tract binding protein (PTB).

### BACKGROUND

Mammalian cells often utilize endogenous alternative splicing mechanisms, whereby multiple mRNAs from a single transcript may be produced. The spliced products may have very different, or even conflicting, functions. Alternative splicing may produce splice variants with differential functions, which may be critical for cellular development and/or homeostasis, and/or intra- and intercellular communication. In addition, modulating splicing regulation can result in consequences related to human disease, such as cancer.

PTB is an RNA binding protein with multiple functions in the regulation of RNA processing and internal ribosomal entry site (IRES)-mediated translation. PTB was originally identified as a protein that bound to the pyrimidine-rich region within introns. PTB is a negative regulator of pre-mRNA splicing by blocking the inclusion of numerous alternative exons into mRNA. Accordingly, PTB expression and activity provides a nexus between disease and cellular mechanisms related to the removal of introns from mRNA precursors.

### SUMMARY OF THE INVENTION

Provided herein is a method for screening for a modulator of PTB. An identified modulator may inhibit or induce PTB activity. The method may comprise providing a cell that comprises PTB and a reporter system. The cell may be mammalian or non-mammalian. The PTB may be endogenously expressed and/or expressed from a heterologous nucleic acid. The heterologous nucleic acid may be a vector. The reporter system may comprise a first PTB target gene operably linked to a reporter sequence. A candidate modulator compound may be contacted with the cell, or the cell may be contacted with the modulator compound. The candidate modulator may be from a library of compounds. The library of compounds may be selected from the group consisting of a peptide library, a natural products library, a cDNA library, a combinatorial library, an oligosaccharide library, a drug library, phage display library, and a small molecule library. The compound may be expressed in the cell. The level of expression of the PTB target gene may be measured. A modulator of PTB may be identified by a change in expression of the PTB target as compared to a control. The first PTB target gene may be a minigene. The PTB target gene may encode a protein selected from the group consisting of c-src, α-actinin, FGF-R2, calcitonin/CGRP, GABA_{A}γ2, α-tropomyosin, PTB1, PTB2, PTB4, GABBR1, INHBE, PICALM, GARNL1, MEIS1, NUMB, PPP3CB, and/or PTBP2. The minigene may encode a fragment of c-src, α-actinin, FGF-R2, calcitonin/CGRP, GABA_{A}γ2, α-tropomyosin, PTB1, PTB2, PTB4, GABBR1, INHBE, PICALM, GARNL1, MEIS1, NUMB, PPP3CB, and/or PTBP2. The minigene may comprise exon 9 to exon 11 of PTBP2. The minigene may comprise exon 14 to exon 16 of GABBR1. The first gene or minigene may be operably linked to a reporter. The first minigene comprising exon 9 to exon 11 of PTBP2 may further comprise exon 11 operably linked to a first reporter. The first minigene comprising exon 14 to exon 16 of GABBR1 may further comprise exon 16 operably linked to a first reporter.

The reporter system may further comprise a second PTB target gene or minigene. The second PTB target gene may encode a protein selected from the group consisting of c-src, α-actinin, FGF-R2, calcitonin/CGRP, GABA_{A}γ2, α-tropomyosin, PTB1, PTB2, PTB4, GABBR1, INHBE, PICALM, GARNL1, MEIS1, NUMB, PPP3CB, and/or PTBP2. The second minigene may encode a fragment of c-src, α-actinin, FGF-R2, calcitonin/CGRP, GABA_{A}γ2, α-tropomyosin, PTB1, PTB2, PTB4, GABBR1, INHBE, PICALM, GARNL1, MEIS1, NUMB, PPP3CB, and/or PTBP2. The second minigene may comprise exon 9 to exon 11 of PTBP2. The second minigene may comprise exon 14 to exon 16 of GABBR1. The second gene or minigene may be operably linked to a reporter. The second minigene comprising exon 9 to exon 11 of PTBP2 may further comprise exon 11 operably linked to a second reporter. The second minigene comprising exon 14 to exon 16 of GABBR1 may further comprise exon 16 operably linked to a second reporter.

The first or second reporter may not be functionally expressed when exon 10 of PTBP2 or exon 15 of GABBR1 is skipped from the transcript of the first or second PTB target, respectively. The first or second reporter may be functionally expressed when exon 10 of PTBP2 or exon 15 of GABBR1 is spliced away from the transcript of the first or second PTB target, respectively. The first or second reporter may be functionally expressed when exon 10 of PTBP2 or exon 15 of GABBR1 is included in the transcript of the first or second PTB target, respectively. The first or second reporter may not be functionally expressed when exon 10 of PTBP2 or exon 15 of GABBR1 is included in the transcript of the first or second PTB target, respectively.

The level of expression of a PTB target may be measured by the level of PTB target gene encoded mRNA. The level of PTB target gene encoded mRNA may be measured by RT-PCR. The level of expression may be measured by reporter output. The reporter output may be fluorescence.

The control may be a cell. The control cell may comprise PTB and the reporter system. The cell may be contacted with a modulator compound that induces, or suppresses, or inhibits, or inhibits completely, PTB-expression and/or activity. The level(s) of expression and/or activity of PTB in the cell in contact with, or formerly in contact with, may be compared to the level(s) of expression and/or activity of PTB in the control cell.

Also provided herein is a method for treating a subject diagnosed with a disease. The method may comprise administering the PTB modulator compound identified by the method described herein to a subject in need thereof. The disease may be cancer. The cancer may be ovarian cancer. The subject may be a mammal. The mammal may be a human.

### BRIEF DESCRIPTION OF THE DRAWINGS

FIG. 1 shows immunoblot analysis of PTB expression in cell lines.

FIG. 2 shows the effects of PTB knockdown using a vector-based DOX (doxycycline)-inducible PTB siRNA.

FIG. 3 shows a tumor growth curve in a xenograft mouse model.

FIG. 4 shows validation of microarray analysis results related to altered expression and altered splicing pattern.

FIG. 5 shows a schematic drawing of a reporter system for detection of PTB activity.

FIG. 6 shows a schematic drawing of lentiviral vectors for use in a reporter system.

FIG. 7 shows a schematic drawing of lentiviral vector expressing DOX-inducible siRNA.

FIG. 8 shows diagram of changes in fluorescent intensity after PTB knockdown by DOX-induced PTB siRNA.

FIG. 9 shows regulation of alternative splicing of exon 15 of GABBR1 by PTB.

FIG. 10 shows a schematic drawing of a reporter system using alternative splicing of GABBR1.

FIG. 11 shows detection of short and long-form SVs of GABBR1 by fluorescent proteins.

### DETAILED DESCRIPTION

The inventors have made the surprising discovery that altered expression and splicing activity of polypyrimidine-tract binding (PTB) protein may be directly related to disease. The ability to identify compounds that modulate PTB expression and/or activity may be useful as a therapeutic for treating a subject having a disease, or predisposed to a disease, as many diseases may be related to alterations in the regulation of splicing of PTB target nucleic acids.

The methods and materials described herein use a PTB and PTB-targeted nucleic acid sequences in a reporter system to recapitulate a splicing pathway in a cell. The splicing pathway may be induced and compounds measured for their effect on PTB and its splicing-related activity.

### 1. DEFINITIONS

The terminology used herein is for the purpose of describing particular embodiments only and is not intended to be limiting. As used in the specification and the appended claims, the singular forms “a,” “an” and “the” include plural references unless the context clearly dictates otherwise.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The numbering of PTBP2 exons used herein corresponds to PTBP2 mRNA of accession number NM_{—}021190. The numbering of GABBR1 exons used herein is based on the GABBR1 mRNA of accession number NM_{—}001470.

a. Fragment

“Fragment” as used herein may mean a portion of a reference peptide or polypeptide or nucleic acid sequence.

b. Identical

“Identical” or “identity” as used herein in the context of two or more polypeptide or nucleotide sequences, may mean that the sequences have a specified percentage of residues or nucleotides that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation.

c. Operably Linked

“Operably linked” as used herein may mean a functional linkage between two polynucleotides, for example a first polynucleotide and a second polynucleotide, wherein expression of one polynucleotide affects transcription and/or translation of the other polynucleotide.

d. Skipped From

“Skipped from” as used herein may mean “not included in” or “spliced away.”

e. Substantially Complementary

“Substantially complementary” as used herein may mean that a first sequence is at least 50%, 55%, 60%, 65%, 70%, 75%, 80%, 85%, 90%, 95%, 96%, 97%, 98%, or 99% identical to the complement of a second sequence over a a region of 8, 9, 10, 20, 30, 40, 50, 60, 70, 80, 90, 100, 150, 200, 250, 300, 350 or more nucleotides or amino acids. Intermediate lengths may mean any length between the quoted values, such as 16, 17, 18, 19, etc.; 21, 22, 23, etc.; 30, 31, 32, etc.; 50, 51, 52, 53, etc.; 100, 101, 102, 103, etc.; 150, 151, 152, 153, etc.; including all integers through 200-500; 500-1,000, and the like.

f. Substantially Identical

“Substantially identical” as used herein may mean that a first and second nucleotide or amino acid sequence are at least about 70%, 75%, 80%, 85%, 90%, 91%, 92%, 93%, 94%, 95%, 96%, 97%, 98%, or 99% identical over a region of 8, 9, 10, 20, 30, 40, 50, 60, 70, 80, 90, 100, 150, 200, 250, 300, 350 or more nucleotides or amino acids. Intermediate lengths may mean any length between the quoted values, such as 16, 17, 18, 19, etc.; 21, 22, 23, etc.; 30, 31, 32, etc.; 50, 51, 52, 53, etc.; 100, 101, 102, 103, etc.; 150, 151, 152, 153, etc.; including all integers through 200-500; 500-1,000, and the like. Substantially identical may also mean the first sequence nucleotide or amino acid sequence is substantially complementary to the complement of the second sequence.

g. Variant

“Variant” as used herein in the context of a nucleic acid may mean a substantially identical or substantially complementary sequence. A variant in reference to a nucleic acid may further mean a nucleic acid that may contain one or more substitutions, additions, deletions, insertions, or may be fragments thereof. A variant may also be a nucleic acid capable of hybridizing under moderately stringent conditions and specifically binding to a nucleic acid encoding the agent. Hybridization techniques are well known in the art and may be conducted under moderately stringent conditions.

A variant in reference to a peptide may further mean differing from a native peptide in one or more substitutions, deletions, additions and/or insertions, or a sequence substantially identical to the native peptide sequence. The ability of a variant to react with antigen-specific antisera may be enhanced or unchanged, relative to the native protein, or may be diminished by less than 50%, or less than 20%, relative to the native peptide. Such variants may generally be identified by modifying one of the peptide sequences encoding an agent and evaluating the reactivity of the modified peptide with antigen-specific antibodies or antisera as described herein. Variants may include those in which one or more portions have been removed such as an N-terminal leader sequence or transmembrane domain. Other variants may include variants in which a small portion (e.g., 1-30 amino acids, or 5-15 amino acids) has been removed from the N- and/or C-terminal of the mature protein.

A variant in reference to a peptide may contain conservative substitutions. A “conservative substitution” is one in which an amino acid is substituted for another amino acid that has similar properties, such that one skilled in the art of peptide chemistry may expect the secondary structure and hydrophobic nature of the polypeptide to be substantially unchanged. Amino acid substitutions may generally be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gin, asn, ser, thr; (2) cys, ser, tyr, thr; (3) val, ile, leu, met, ala, phe; (4) lys, arg, his; and (5) phe, tyr, trp, his. A variant may also contain nonconservative changes. Variant peptides may differ from a native sequence by substitution, deletion or addition of amino acids. Variants may also be modified by deletion or addition of amino acids, which have minimal influence on the immunogenicity, secondary structure, hydropathic, and hydrophobic nature of the polypeptide.

### 2. METHOD OF IDENTIFYING MODULATORS OF PTB

Provided herein is a method of screening for modulators of PTB. A cell may be provided that comprises PTB and a reporter system, which may comprise a first PTB target gene operably linked to a first reporter sequence. The reporter system may further comprise a second PTB target gene operably linked to a second reporter sequence.

The cell may be contacted with a candidate modulator compound. After contact by the candidate compound, the level of expression of the PTB target gene may be measured. A modulator of PTB may be identified by a change in expression of the PTB target gene compared to a control. The level of expression of the PTB target gene may be measured as mRNA and/or protein.

a. PTB

Polypyrimidine-tract binding protein (PTB) may be any mammalian PTB protein as well as variants thereof. The mammalian PTB may be human. Representative examples of PTB include those shown in Table 1.

TABLE 1

| Organism | Gene and Accession No. |
|---|---|
| human | PTBP1 isoform a (NCBI accession no. |
| | NP_002810), |
| | PTBP1 isoform b (NCBI accession no. |
| | NP_114367), |
| | PTBP1 isoform c (NCBI accession no. |
| | NP_114366), and/or |
| | PTBP1 isoform d (NCBI accession no. |
| | NP_787041) |
| S. scrofa | PTB4, NCBI protein accession no. CAA63597 |
| Mus musculis | PTB1, NCBI protein accession no. P17225 |
| R. norvegicus | PTB4, NCBI protein accession no. Q00438 |
| X. laevis | PTB4, NCBI protein accession no. AAF00041 |
| D. melanogaster | PTB4, NCBI protein accession no. AAF22979 |
| C. elegans | PTB4, NCBI protein accession no. T20381 |
| Danio rerio | ptbp1, NCBI accession no. NP_001018313 |
| Gallus gallus | PTBP1, NCBI accession no. NP_001026106 |
| Xenopus (Silurana) | ptbp1, NCBI aqccession no. NP_001011140 |
| tropicalis | |
| Pan troglodytes | PTBP1, NCBI accession no. XP_001172084 |
| Equus caballus | PTB1, NCBI accession no. XP_001915461 |
| Macaca mulatto | PTBP1 isoform 1, NBI accession no. |
| | XP_001092088 |
| Canis lupus familiaris | PTBP1, NCBI accession no. XP_868639 |
| Bos taurus | PTBP1, NCBI accession no. NP_776867 |

PTB may be expressed from a cell chromosome and/or a heterologous nucleic acid. The heterologous nucleic acid may be a vector or plasmid. PTB expression is typically directed by a promoter. A promoter can be naturally associated with a nucleic acid sequence, as can be obtained by isolating the 5′ non-coding sequences located upstream of the coding segment and/or exon. Alternatively, the promoter can be from a different gene, or from a gene from a different species of organism (“heterologous”). Expression of PTB may be controlled by an inducible promoter, such as, for example, Gal1-10, Gal1, GalL, GalS, or CUP1 or a repressible promoter, such as Met25, for expression in yeast. An inducible promoter may be active under environmental or developmental regulation. The inducible promoter may be capable of functioning in a eukaryotic host organism. These promoters include naturally occurring yeast and mammalian inducible promoters as well as synthetic promoters designed to function in a eukaryotic host. An important functional characteristic of an inducible promoter is its inducibility by exposure to an environmental inducing agent. Appropriate environmental inducing agents include exposure to heat, various steroidal compounds, divalent cations (including Cu⁺² and Zn⁺²), galactose, tetracycline, IPTG (isopropyl-β-D thiogalactoside), as well as other naturally occurring and synthetic inducing agents and gratuitous inducers. The inducible promoter may be a vector-based DOX (doxycycline)-inducible promoter. Synthetic inducible promoter systems are also available for use. Suitable expression cassettes are readily available for heterologous expression in many different eukaryotic cells including various yeast species and mammalian cells. PTB nucleic acids can include at least one termination signal and/or polyadenylation signal, as needed.

b. Reporter System

(1) PTB Target Gene

The reporter system may comprise one or more PTB target genes. For example, there may be at least 1, 2, 3, 4, 5, 6, 7, or more target genes. Any of the PTB target genes may be expressed from a heterologous nucleic acid. The heterologous nucleic acid may be a vector or plasmid. The PTB target genes may be expressed from two or more separate vectors or plasmids. The one or more PTB target genes may be nucleotide sequences. The nucleotide sequences may be full-length pre-mRNA nucleotide sequences of the PTB target or variants thereof. The one or more PTB target genes may be pre-mRNA sequences encoded by one or more genes. The PTB target pre-mRNA sequence may be spliced via a PTB-driven mechanism. The gene may encode c-src, α-actinin, FGF-R2, calcitonin/CGRP, GABA_{A}γ2, α-tropomyosin, PTB1, PTB2, PTB4, GABBR1, INHBE, PICALM, GARNL1, MEIS1, NUMB, PPP3CB, and/or PTBP2. The gene may be one or more genes selected from Table 2.

The one or more PTB target genes may be pre-mRNA sequences encoded by one or more minigenes. The PTB target pre-mRNA sequence may be spliced via a PTB-driven mechanism. The minigene may be a fragment of a gene or a variant thereof. Any of the PTB target minigenes may be expressed from a heterologous nucleic acid. The heterologous nucleic acid may be a vector or plasmid. The PTB target minigenes may be expressed from two or more separate vectors or plasmids. A minigene may comprise a genomic sequence spanning one or more, two or more, three or more, four or more, or five or more exons of the PTB target. A minigene may comprise a genomic sequence spanning one or more, two or more, three or more, four or more, or five or more introns of a PTB target gene. A minigene may comprise a genomic sequence comprising any number of introns or exons of a PTB target. The minigene may comprise a genomic sequence comprising exons 1 through 3, 2 through 4, 4 through 6, 5 through 7, 6 through 8, 7 through 9, 8 through 10, 9 through 11, 10 through 12, 11 through 13, and/or 12 through 14 of PTBP2. The minigene may comprise a genomic sequence comprising exons 1 through 3, 2 through 4, 4 through 6, 5 through 7, 6 through 8, 7 through 9, 8 through 10, 9 through 11, 10 through 12, 13 through 15, 14 through 16, 15 through 17, 16 through 18, 17 through 19, 18 through 20, 19 through 21, 20 through 22, and/or 21 through 23 of GABBR1. The minigene may encode a fragment of c-src, α-actinin, FGF-R2, calcitonin/CGRP, GABA_{A}γ2, α-tropomyosin, PTB1, PTB2, PTB4, GABBR1, INHBE, PICALM, GARNL1, MEIS1, NUMB, PPP3CB, and/or PTBP2. The minigene may encode a fragment of one or more proteins encoded by the genes selected from Table 2.

TABLE 2
PTB-regulated genes

| GENE_SYMBOL | Gene Name |
|---|---|
| STX3A | syntaxin 3a |
| CSDE1 | cold shock domain containing e1, rna-binding |
| CCNB1IP1 | cyclin b1 interacting protein 1 |
| ANKRD10 | ankyrin repeat domain 10 |
| TSC22D3 | tsc22 domain family, member 3 |
| PPP3CB | protein phosphatase 3 (formerly 2b), catalytic |
| | subunit, beta isoform (calcineurin a beta) |
| EXOC7 | exocyst complex component 7 |
| EXO1 | exonuclease 1 |
| TDRD3 | tudor domain containing 3 |
| TPM2 | tropomyosin 2 (beta) |
| LOC55565 | hypothetical protein loc55565 |
| SEC13L1 | sec13-like 1 (s. cerevisiae) |
| TMEM16C | transmembrane protein 16c |
| NFYC | nuclear transcription factor y, gamma |
| EHBP1 | eh domain binding protein 1 |
| MLLT10 | myeloid/lymphoid or mixed-lineage leukemia |
| | (trithorax homolog, drosophila); translocated |
| | to, 10 |
| TSC2 | tuberous sclerosis 2 |
| USP54 | ubiquitin specific peptidase 54 |
| CBS | cystathionine-beta-synthase |
| PCDH17 | protocadherin 17 |
| ARRB1 | arrestin, beta 1 |
| LRRFIP2 | leucine rich repeat (in flii) interacting protein 2 |
| CDK5RAP2 | cdk5 regulatory subunit associated protein 2 |
| BMPR2 | bone morphogenetic protein receptor, type ii |
| | (serine/threonine kinase) |
| KCNK2 | potassium channel, subfamily k, member 2 |
| KPNA1 | karyopherin alpha 1 (importin alpha 5) |
| ABI2 | abl interactor 2 |
| SCARB1 | scavenger receptor class b, member 1 |
| AMT | aminomethyltransferase (glycine cleavage |
| | system protein t) |
| GOLPH4 | golgi phosphoprotein 4 |
| MYO5A | myosin va (heavy polypeptide 12, myoxin) |
| C14orf118 | chromosome 14 open reading frame 118 |
| TPM1 | tropomyosin 1 (alpha) |
| RNPC2 | rna-binding region (rnp1, rrm) containing 2 |
| NCOA7 | nuclear receptor coactivator 7 |
| PPP3CA | protein phosphatase 3 (formerly 2b), catalytic |
| | subunit, alpha isoform (calcineurin a alpha) |
| USP16 | ubiquitin specific peptidase 16 |
| SCP2 | sterol carrier protein 2 |
| CATSPER2 | cation channel, sperm associated 2 |
| MCM7 | mcm7 minichromosome maintenance deficient |
| | 7 (s. cerevisiae) |
| NYREN18 | nedd8 ultimate buster-1 |
| OCRL | oculocerebrorenal syndrome of lowe |
| OGT | o-linked n-acetylglucosamine (glcnac) |
| | transferase (udp-n- |
| | acetylglucosamine:polypeptide-n- |
| | acetylglucosaminyl transferase) |
| NEDD4L | neural precursor cell expressed, |
| | developmentally down-regulated 4-like |
| STOX2 | storkhead box 2 |
| SFRS2 | splicing factor, arginine/serine-rich 2 |
| NFATC4 | nuclear factor of activated t-cells, cytoplasmic, |
| | calcineurin-dependent 4 |
| HMGCL | 3-hydroxymethyl-3-methylglutaryl-coenzyme |
| | a lyase (hydroxymethylglutaricaciduria) |
| 39331 | septin 6 |
| SH3KBP1 | sh3-domain kinase binding protein 1 |
| ATP9B | atpase, class ii, type 9b |
| SYNE1 | spectrin repeat containing, nuclear envelope 1 |
| SMG7 | smg-7 homolog, nonsense mediated mrna |
| | decay factor (c. elegans) |
| SEPP1 | selenoprotein p, plasma, 1 |
| PICALM | phosphatidylinositol binding clathrin assembly |
| | protein |
| PTPN13 | protein tyrosine phosphatase, non-receptor type |
| | 13 (apo-1/cd95 (fas)-associated phosphatase) |
| CDC42BPA | cdc42 binding protein kinase alpha (dmpk-like) |
| ATP11C | atpase, class vi, type 11c |
| CTNNB1 | catenin (cadherin-associated protein), beta 1, |
| | 88 kda |
| TSPAN1 | tetraspanin 1 |
| C20orf96 | chromosome 20 open reading frame 96 |
| PPA2 | pyrophosphatase (inorganic) 2 |
| EPB41 | erythrocyte membrane protein band 4.1 |
| | (elliptocytosis 1, rh-linked) |
| ARHGEF7 | rho guanine nucleotide exchange factor (gef) 7 |
| DPYD | dihydropyrimidine dehydrogenase |
| DZIP1 | daz interacting protein 1 |
| MCM10 | mcm10 minichromosome maintenance |
| | deficient 10 (s. cerevisiae) |
| ISL1 | isl1 transcription factor, lim/homeodomain, |
| | (islet-1) |
| WDR45L | wdr45-like |
| POU1F1 | pou domain, class 1, transcription factor 1 |
| | (pit1, growth hormone factor 1) |
| GDPD1 | glycerophosphodiester phosphodiesterase |
| | domain containing 1 |
| LMAN2L | lectin, mannose-binding 2-like |
| FLJ10357 | hypothetical protein flj10357 |
| DDIT3 | dna-damage-inducible transcript 3 |
| NOX4 | nadph oxidase 4 |
| TXNIP | thioredoxin interacting protein |
| GABBR1 | gamma-aminobutyric acid (gaba) b receptor, 1 |
| LOC153222 | adult retina protein |
| LETMD1 | letm1 domain containing 1 |
| LRRFIP1 | leucine rich repeat (in flii) interacting protein 1 |
| MEF2D | mads box transcription enhancer factor 2, |
| | polypeptide d (myocyte enhancer factor 2d) |
| UROD | uroporphyrinogen decarboxylase |
| NUMB | numb homolog (drosophila) |
| AK2 | adenylate kinase 2 |
| SWAP70 | swap-70 protein |
| RAPGEF2 | rap guanine nucleotide exchange factor (gef) 2 |
| TTLL6 | tubulin tyrosine ligase-like family, member 6 |
| CTSC | cathepsin c |
| RSN | restin (reed-steinberg cell-expressed |
| | intermediate filament-associated protein) |
| MAPK10 | mitogen-activated protein kinase 10 |
| ZDHHC6 | zinc finger, dhhc-type containing 6 |
| KNS2 | kinesin 2 |
| TEX9 | testis expressed sequence 9 |
| MEF2A | mads box transcription enhancer factor 2, |
| | polypeptide a (myocyte enhancer factor 2a) |
| TPM3 | tropomyosin 3 |
| PIK3C3 | phosphoinositide-3-kinase, class 3 |
| CSTA | cystatin a (stefin a) |
| OCA2 | oculocutaneous albinism ii (pink-eye dilution |
| | homolog, mouse) |
| BAG1 | bcl2-associated athanogene |
| RPS24 | ribosomal protein s24 |
| HPS4 | le protein |
| CTH | cystathionase (cystathionine gamma-lyase) |
| KIFAP3 | kinesin-associated protein 3 |
| YPEL5 | yippee-like 5 (drosophila) |
| EXOSC3 | exosome component 3 |
| SSFA2 | sperm specific antigen 2 |
| CLTC | clathrin, heavy polypeptide (hc) |
| ERBB2IP | erbb2 interacting protein |
| C20orf19 | chromosome 20 open reading frame 19 |
| MERTK | c-mer proto-oncogene tyrosine kinase |
| WDR6 | wd repeat domain 6 |
| ANGEL2 | angel homolog 2 (drosophila) |
| CREB3L1 | camp responsive element binding protein 3- |
| | like 1 |
| CARM1 | coactivator-associated arginine |
| | methyltransferase 1 |
| WDR25 | wd repeat domain 25 |
| TSLP | thymic stromal lymphopoietin |
| CALD1 | caldesmon 1 |
| C2orf13 | chromosome 2 open reading frame 13 |
| RECK | reversion-inducing-cysteine-rich protein with |
| | kazal motifs |
| COMMD4 | comm domain containing 4 |
| ABLIM1 | actin binding lim protein 1 |
| CARS | cysteinyl-trna synthetase |
| PHF14 | phd finger protein 14 |
| ATXN7 | ataxin 7 |
| PTBP2 | polypyrimidine tract binding protein 2 |
| DDIT4 | dna-damage-inducible transcript 4 |
| PRSS16 | protease, serine, 16 (thymus) |
| COPS5 | cop9 constitutive photomorphogenic homolog |
| | subunit 5 (arabidopsis) |
| ASPH | aspartate beta-hydroxylase |
| CCT4 | chaperonin containing tcp1, subunit 4 (delta) |
| PRSS7 | protease, serine, 7 (enterokinase) |
| PIGX | phosphatidylinositol glycan, class x |
| COG3 | component of oligomeric golgi complex 3 |
| LASS5 | lag1 longevity assurance homolog 5 |
| | (s. cerevisiae) |
| IL21R | interleukin 21 receptor |
| B4GALT4 | udp-gal:betaglcnac beta 1,4- |
| | galactosyltransferase, polypeptide 4 |
| TRERF1 | transcriptional regulating factor 1 |
| RAPH1 | ras association (ralgds/af-6) and pleckstrin |
| | homology domains 1 |
| LYK5 | protein kinase lyk5 |
| MPZL1 | myelin protein zero-like 1 |
| USP28 | ubiquitin specific peptidase 28 |
| KIAA1109 | kiaa1371 protein |
| VMD2 | vitelliform macular dystrophy 2 (best disease, |
| | bestrophin) |
| ROBO3 | roundabout, axon guidance receptor, homolog |
| | 3 (drosophila) |
| SMNDC1 | survival motor neuron domain containing 1 |
| DLD | dihydrolipoamide dehydrogenase (e3 |
| | component of pyruvate dehydrogenase |
| | complex, 2-oxo-glutarate complex, branched |
| | chain keto acid dehydrogenase complex) |
| CRYZ | crystallin, zeta (quinone reductase) |
| IQGAP1 | iq motif containing gtpase activating protein 1 |
| SLC37A4 | solute carrier family 37 (glycerol-6-phosphate |
| | transporter), member 4 |
| TNRC6A | trinucleotide repeat containing 6a |
| PLEKHH2 | pleckstrin homology domain containing, |
| | family h (with myth4 domain) member 2 |
| PLD1 | phospholipase d1, phosphatidylcholine-specific |
| PKP4 | plakophilin 4 |
| ARHGEF2 | rho/rac guanine nucleotide exchange factor |
| | (gef) 2 |
| ITPA | inosine triphosphatase (nucleoside triphosphate |
| | pyrophosphatase) |
| C3orf17 | chromosome 3 open reading frame 17 |
| CBLL1 | cas-br-m (murine) ecotropic retroviral |
| | transforming sequence-like 1 |
| THOC5 | tho complex 5 |
| LOC81691 | exonuclease nef-sp |
| AP1GBP1 | ap1 gamma subunit binding protein 1 |
| ADD1 | adducin 1 (alpha) |
| PTBP1 | polypyrimidine tract binding protein 1 |
| TCF7 | transcription factor 7 (t-cell specific, hmg-box) |
| NIPBL | nipped-b homolog (drosophila) |
| PFN2 | profilin 2 |
| CLUAP1 | clusterin associated protein 1 |
| ME1 | malic enzyme 1, nadp(+)-dependent, cytosolic |
| JDP2 | jun dimerization protein 2 |
| SVIL | supervillin |
| ERO1LB | ero1-like beta (s. cerevisiae) |
| MDS025 | hypothetical protein mds025 |
| DHRS2 | dehydrogenase/reductase (sdr family) member 2 |
| DTX2 | kiaa1528 protein |
| NRG4 | neuregulin 4 |
| FANCA | fanconi anemia, complementation group a |
| NOL5A | nucleolar protein 5a (56 kda with kke/d repeat) |
| ATP6V1H | atpase, h+ transporting, lysosomal 50/57 kda, |
| | v1 subunit h |
| ACTN1 | actinin, alpha 1 |
| CRSP3 | cofactor required for sp1 transcriptional |
| | activation, subunit 3, 130 kda |
| SEC61A2 | sec61 alpha 2 subunit (s. cerevisiae) |
| AZIN1 | antizyme inhibitor 1 |
| MRPS22 | mitochondrial ribosomal protein s22 |
| ITGA5 | integrin, alpha 5 (fibronectin receptor, alpha |
| | polypeptide) |
| MAP4 | microtubule-associated protein 4 |
| DCDC2 | doublecortin domain containing 2 |
| GFPT1 | glutamine-fructose-6-phosphate transaminase 1 |
| GMPR2 | guanosine monophosphate reductase 2 |
| ADHFE1 | alcohol dehydrogenase, iron containing, 1 |
| INHBE | inhibin, beta e |
| EPB41L1 | erythrocyte membrane protein band 4.1-like 1 |
| MAP3K4 | mitogen-activated protein kinase kinase kinase 4 |
| ACSM3 | acyl-coa synthetase medium-chain family |
| | member 3 |
| ECHDC1 | enoyl coenzyme a hydratase domain containing 1 |
| TTC13 | tetratricopeptide repeat domain 13 |
| KLHDC1 | kelch domain containing 1 |
| KIAA1377 | kiaa1377 |
| UBE2V2 | ubiquitin-conjugating enzyme e2 variant 2 |
| ASS | argininosuccinate synthetase |
| SLC38A6 | solute carrier family 38, member 6 |
| MRPS27 | mitochondrial ribosomal protein s27 |
| TTC9C | tetratricopeptide repeat domain 9c |
| NEK7 | nima (never in mitosis gene a)-related kinase 7 |
| EAF1 | ell associated factor 1 |
| ITGB3BP | integrin beta 3 binding protein (beta3- |
| | endonexin) |
| TRIM2 | tripartite motif-containing 2 |
| FNDC6 | fibronectin type iii domain containing 6 |
| C21orf6 | chromosome 21 open reading frame 6 |
| SFRS14 | splicing factor, arginine/serine-rich 14 |
| CBX5 | chromobox homolog 5 (hp1 alpha homolog, |
| | drosophila) |
| ATP1B3 | atpase, na+/k+ transporting, beta 3 polypeptide |
| MOV10 | mov10, moloney leukemia virus 10, homolog |
| | (mouse) |
| PRR3 | proline rich 3 |
| SLC7A11 | solute carrier family 7, (cationic amino acid |
| | transporter, y+ system) member 11 |
| MRPL20 | mitochondrial ribosomal protein 120 |
| PBX1 | pre-b-cell leukemia transcription factor 1 |
| CASP2 | caspase 2, apoptosis-related cysteine peptidase |
| | (neural precursor cell expressed, |
| | developmentally down-regulated 2) |
| ANK3 | ankyrin 3, node of ranvier (ankyrin g) |
| CYP4V2 | cytochrome p450, family 4, subfamily v, |
| | polypeptide 2 |
| CTAGE5 | ctage family, member 5 |
| PDLIM7 | pdz and lim domain 7 (enigma) |
| STRA6 | stimulated by retinoic acid gene 6 homolog |
| | (mouse) |
| IKIP | ikk interacting protein |
| CCPG1 | cell cycle progression 1 |
| FBXO38 | hypothetical protein flj13962 |
| PRKX | protein kinase, x-linked |
| TNIK | traf2 and nck interacting kinase |
| ASNS | asparagine synthetase |
| RBM25 | rna binding motif protein 25 |
| HIST1H2BD | histone 1, h2bd |
| KIAA0528 | kiaa0528 |
| SFRS10 | splicing factor, arginine/serine-rich 10 |
| | (transformer 2 homolog, drosophila) |
| PTPDC1 | protein tyrosine phosphatase domain |
| | containing 1 |
| COL5A2 | collagen, type v, alpha 2 |
| AGA | aspartylglucosaminidase |
| SFRS3 | splicing factor, arginine/serine-rich 3 |
| HDAC6 | histone deacetylase 6 |
| BTG1 | b-cell translocation gene 1, anti-proliferative |
| SNX14 | sorting nexin 14 |
| ARFIP1 | adp-ribosylation factor interacting protein 1 |
| | (arfaptin 1) |
| XBP1 | x-box binding protein 1 |
| PPFIA1 | protein tyrosine phosphatase, receptor type, f |
| | polypeptide (ptprf), interacting protein (liprin), |
| | alpha 1 |
| IFRD1 | interferon-related developmental regulator 1 |
| SNAP23 | synaptosomal-associated protein, 23 kda |
| SH3YL1 | sh3 domain containing, ysc84-like 1 |
| | (s. cerevisiae) |
| CAPZB | capping protein (actin filament) muscle z-line, |
| | beta |
| CSPG5 | chondroitin sulfate proteoglycan 5 |
| | (neuroglycan c) |
| LAS1L | las1-like (s. cerevisiae) |
| CCNA2 | cyclin a2 |
| GDAP1L1 | ganglioside-induced differentiation-associated |
| | protein 1-like 1 |
| DCTN1 | dynactin 1 (p150, glued homolog, drosophila) |
| HIPK3 | homeodomain interacting protein kinase 3 |
| NUP62 | nucleoporin 62 kda |
| NECAP1 | necap endocytosis associated 1 |
| NDUFV3 | nadh dehydrogenase (ubiquinone) flavoprotein |
| | 3, 10 kda |
| SLCO4A1 | solute carrier organic anion transporter family, |
| | member 4a1 |
| CAMKK2 | calcium/calmodulin-dependent protein kinase |
| | kinase 2, beta |
| DLG2 | discs, large homolog 2, chapsyn-110 |
| | (drosophila) |
| TBC1D23 | tbc1 domain family, member 23 |
| SNX13 | sorting nexin 13 |
| ATP2B4 | atpase, ca++ transporting, plasma membrane 4 |
| AURKB | aurora kinase b |
| CDC25A | cell division cycle 25a |
| FBXO25 | f-box protein 25 |
| SAP18 | sin3a-associated protein, 18 kda |
| MMP10 | matrix metallopeptidase 10 (stromelysin 2) |
| TRAPPC4 | trafficking protein particle complex 4 |
| ZNF195 | zinc finger protein 195 |
| KTN1 | kinectin 1 (kinesin receptor) |
| CPEB1 | cytoplasmic polyadenylation element binding |
| | protein 1 |
| ODF2 | outer dense fiber of sperm tails 2 |
| EFEMP2 | fibulin 4 |
| PCM1 | pericentriolar material 1 |
| KIAA0494 | kiaa0494 |
| DNAJC3 | dnaj (hsp40) homolog, subfamily c, member 3 |
| FEZ2 | fasciculation and elongation protein zeta 2 |
| | (zygin ii) |
| SRRM1 | serine/arginine repetitive matrix 1 |
| TFRC | transferrin receptor (p90, cd71) |
| KIFC1 | kinesin family member c1 |
| OSBPL9 | hypothetical protein flj12492 |
| ARHGEF11 | rho guanine nucleotide exchange factor (gef) |
| | 11 |
| ZMYND12 | zinc finger, mynd-type containing 12 |
| MEIS1 | meis1, myeloid ecotropic viral integration site |
| | 1 homolog (mouse) |
| FUSIP1 | fus interacting protein (serine/arginine-rich) 1 |
| CALCRL | calcitonin receptor-like |
| LEPRE1 | leucine proline-enriched proteoglycan |
| | (leprecan) 1 |
| VLDLR | very low density lipoprotein receptor |
| APP | amyloid beta (a4) precursor protein (peptidase |
| | nexin-ii, alzheimer disease) |
| FGG | fibrinogen gamma chain |
| TUG1 | taurine upregulated gene 1 |
| S100A4 | s100 calcium binding protein a4 (calcium |
| | protein, calvasculin, metastasin, murine |
| | placental homolog) |
| NDUFS7 | nadh dehydrogenase (ubiquinone) fe—s protein |
| | 7, 20 kda (nadh-coenzyme q reductase) |
| ATF3 | activating transcription factor 3 |
| LPHN2 | latrophilin 2 |
| SLC25A36 | solute carrier family 25, member 36 |
| TCF20 | transcription factor 20 (ar1) |
| ASF1B | asf1 anti-silencing function 1 homolog b |
| | (s. cerevisiae) |
| ART3 | adp-ribosyltransferase 3 |
| PACSIN2 | protein kinase c and casein kinase substrate in |
| | neurons 2 |
| C6orf142 | chromosome 6 open reading frame 142 |
| ORAOV1 | oral cancer overexpressed 1 |
| TCF7L2 | transcription factor 7-like 2 (t-cell specific, |
| | hmg-box) |
| SLC1A4 | solute carrier family 1 (glutamate/neutral |
| | amino acid transporter), member 4 |
| SEMA3C | sema domain, immunoglobulin domain (ig), |
| | short basic domain, secreted, (semaphorin) 3c |
| WARS | interferon-induced protein 53 |
| ATP2A2 | atpase, ca++ transporting, cardiac muscle, slow |
| | twitch 2 |
| SLC16A6 | solute carrier family 16 (monocarboxylic acid |
| | transporters), member 6 |
| C1orf91 | chromosome 1 open reading frame 91 |
| RWDD1 | rwd domain containing 1 |
| DOCK9 | dedicator of cytokinesis 9 |
| GDAP1 | ganglioside-induced differentiation-associated |
| | protein 1 |
| AKAP13 | lymphoid blast crisis oncogene |
| OSBPL6 | osbp-related protein 6 |
| PDE1A | phosphodiesterase 1a, calmodulin-dependent |
| DDX47 | dead (asp-glu-ala-asp) box polypeptide 47 |
| MAP3K15 | mitogen-activated protein kinase kinase kinase |
| | 15 |
| KRIT1 | krit1, ankyrin repeat containing |
| B3GNT6 | udp-glcnac:betagal beta-1,3-n- |
| | acetylglucosaminyltransferase 6 |
| MBNL2 | muscleblind-like 2 (drosophila) |
| C13orf23 | chromosome 13 open reading frame 23 |
| SPTAN1 | spectrin, alpha, non-erythrocytic 1 (alpha- |
| | fodrin) |
| FMNL2 | formin-like 2 |
| KIF21A | kinesin family member 21a |
| FNDC3A | fibronectin type iii domain containing 3a |
| ARRDC4 | arrestin domain containing 4 |
| TRIM4 | tripartite motif containing 4 |
| ZCCHC8 | zinc finger, cchc domain containing 8 |
| ANXA7 | annexin a7 |
| TRAM1 | translocation associated membrane protein 1 |
| KIAA1411 | kiaa1411 |
| CBLB | cas-br-m (murine) ecotropic retroviral |
| | transforming sequence b |
| SMARCA1 | swi/snf related, matrix associated, actin |
| | dependent regulator of chromatin, subfamily a, |
| | member 1 |
| DNAJC12 | dnaj (hsp40) homolog, subfamily c, member |
| | 12 |
| CAPG | capping protein (actin filament), gelsolin-like |
| POPDC3 | popeye domain containing 3 |
| RBM28 | rna binding motif protein 28 |
| GARNL1 | kiaa0884 protein |
| MARS | methionine-trna synthetase |
| SLC43A1 | solute carrier family 43, member 1 |
| SPAG9 | sperm associated antigen 9 |
| BAT2D1 | bat2 domain containing 1 |
| PPHLN1 | hypothetical protein hspc206 |
| PLCL3 | phospholipase c-like 3 |
| IL6R | interleukin 6 receptor |
| STC2 | stanniocalcin 2 |
| PSAT1 | phosphoserine aminotransferase 1 |
| COL4A3BP | collagen, type iv, alpha 3 (goodpasture antigen) |
| | binding protein |
| GRHL1 | grainyhead-like 1 (drosophila) |
| RNF41 | ring finger protein 41 |
| GPC2 | glypican 2 (cerebroglycan) |
| TUBE1 | tubulin, epsilon 1 |
| SEH1L | seh1-like (s. cerevisiae) |
| BIN1 | bridging integrator 1 |
| NFS1 | nfs1 nitrogen fixation 1 (s. cerevisiae) |
| HRAS | v-ha-ras harvey rat sarcoma viral oncogene |
| | homolog |
| KIAA0350 | kiaa0350 protein |
| CLIC4 | chloride intracellular channel 4 |
| C6orf107 | chromosome 6 open reading frame 107 |
| PKM2 | pyruvate kinase, muscle |
| TMEM45A | transmembrane protein 45a |
| GARS | glycyl-trna synthetase |
| PHF21A | phd finger protein 21a |
| CDC42 | cell division cycle 42 (gtp binding protein, |
| | 25 kda) |
| CEPT1 | choline/ethanolamine phosphotransferase 1 |
| PCK2 | phosphoenolpyruvate carboxykinase 2 |
| | (mitochondrial) |
| C19orf15 | chromosome 19 open reading frame 15 |
| DNAJA1 | dnaj (hsp40) homolog, subfamily a, member 1 |
| RPS6KA2 | ribosomal protein s6 kinase, 90 kda, |
| | polypeptide 2 |
| ZNF584 | zinc finger protein 584 |
| FAM102A | family with sequence similarity 102, member a |
| H1F0 | h1 histone family, member 0 |
| RBM35A | rna binding motif protein 35a |
| BAX | bcl2-associated x protein |
| HERPUD1 | homocysteine-inducible, endoplasmic |
| | reticulum stress-inducible, ubiquitin-like |
| | domain member 1 |
| AHSA2 | aha1, activator of heat shock 90 kda protein |
| | atpase homolog 2 (yeast) |
| TXNL4B | thioredoxin-like 4b |
| RTN4 | reticulon 4 |
| BBX | bobby sox homolog (drosophila) |
| DCC1 | defective in sister chromatid cohesion homolog |
| | 1 (s. cerevisiae) |
| FLJ21908 | hypothetical protein flj21908 |
| TAPBP | tap binding protein (tapasin) |
| KIAA1217 | kiaa1217 |
| RIF1 | dkfzp434d193 protein |
| UBB | ubiquitin b |
| ITSN2 | sh3 domain protein 1b |
| PPIL3 | peptidylprolyl isomerase (cyclophilin)-like 3 |
| BLP1 (TM2D2) | TM2 domain containing 2 |
| C16orf75 | chromosome 16 open reading frame 75 |
| C4orf32 | chromosome 4 open reading frame 32 |
| C6orf80 | chromosome 4 open reading frame 32 |
| CLPTM1L | CLPTM1-like |
| FAM122C | family with sequence similarity 122C |
| FAM129A | family with sequence similarity 129, member A |
| FLJ20171 (RBM35A) | RNA binding motif protein 35A |
| LOC90529 (C1orf201) | chromosome 1 open reading frame 201 |
| MKX | mohawk homeobox |
| NBLA10383 (CDRT4) | CMT1A duplicated region transcript 4 |
| TBC1D9B | TBC1 domain family, member 9B |
| WIPI49 | WD repeat domain, phosphoinositide |
| | interacting 1 |

(2) Reporter

The PTB target may be operably linked to a nucleotide sequence that encodes a reporter. The reporter may be green fluorescent protein (GFP), enhanced green fluorescent protein (EGFP), red fluorescent protein, monomeric red fluorescent protein, cyan fluorescent protein (CFP), yellow fluorescent protein (YFP), bluefluorescent protein (BFP), cycle 3 GFP, Emerald GFP, β-galactosidase, luciferase, chloramphenyl acetyltransferase (CAT), GUS (β-glucuronidase), and/or a variant or fragment thereof.

A first gene, or minigene, target construct may be constructed such that when the gene or minigene is properly spliced, the downstream reporter coding sequence may be in frame. Expression of such a minigene will produce a reporter output signal. However, when a middle exon, exon 10 of a minigene comprising exons 9 through 11 of PTBP2 for example, is spliced out of the transcript, or skipped over, a frameshift may occur such that the downstream reporter coding sequence is out of frame and a reporter output signal will not be produced.

A second gene, or minigene, construct may be constructed such that the downstream reporter is out of frame when all exons are splice together, but are in frame when a middle exon is skipped from the transcript.

An exon may be modified. The modified exon may comprise a frameshifting nucleotide. The frameshifting nucleotide may result in a stop codon that is incorporated into the mRNA sequence. The frameshifting nucleotide may result in a stop codon that is incorporated into the mRNA sequence, if the exon is included in the spliced transcript. The frameshifting nucleotide may result in a stop codon that is incorporated into the mRNA sequence, if the exon is excluded from the spliced transcript.

The gene or minigene may be modified. The reporter may not be expressed if a modified exon is included in the spliced transcript. The modified exon may be upstream of the reporter. The reporter may be expressed, if the modified exon is excluded from the resultant mRNA. Translation of the modified exon comprising a frameshifting nucleotide may result in the reporter not being expressed.

The gene or minigene may be modified such that the reporter may not be expressed if an exon is excluded from the resultant mRNA. The modified minigene may result in a frameshift in a downstream reading frame when the exon is skipped from the resultant mRNA. The frameshift may result in the presence of a stop codon. The modification may be upstream of the reporter. The stop codon may be upstream of the reporter.

c. Candidate Modulator Compound

The method may use a candidate modulator compound. The candidate modulator may be a candidate for modulating PTB. The cell may express the candidate modulator compound, wherein the expressed candidate modulator compound is in contact with the cell. The expressed candidate modulator compound may be expressed and then secreted from the cell. The secreted candidate modulator compound may be in contact with the cell. The method may comprise stimulating the cell to express the candidate modulator compound. The method may cause the cell to take up the candidate modulator compound.

The candidate modulator compound may be expressed from a vector or plasmid. The candidate modulator compound may be expressed from a vector or plasmid in the cell. Expression may be controlled via a promoter. The promoter may be inducible. The expressed candidate modulator compound may be secreted from the cell. The candidate modulator compound may be a member of a library to be screened using the method herein described. The library may be combinatorial. A candidate modulator compound may be an antibody, a small compound or molecule, a drug, a peptide, a nucleic acid, an oligosaccharide, or an inorganic compound. The nucleic acid may be a siRNA.

d. Cell

The cell may be any cell. The cell may be capable of propagating and/or expressing a vector or plasmid. The cell may be eukaryotic or prokaryotic. The eukaryotic or prokaryotic cell may be living. The eukaryotic cell may be mammalian. The mammalian cell may be a HeLa cell, a CHO cell, a human embryonic kidney cell, or a cancer cell. The human embryonic kidney cell may be a HEK 293 cell or a HEK 293T cell. The cancer cell may be an ovarian cancer cell or a breast cancer cell. The mammalian cell may be from, or in, a sample. The sample may be from a subject.

(1) Sample

The sample may be a subject sample and/or a control sample. The sample may comprise nucleic acid and/or protein from a subject. The nucleic acid may be DNA or RNA. The nucleic acid may be genomic. The sample may be used directly as obtained from the subject or following pretreatment to modify a character of the sample. Pretreatment may include extraction, concentration, inactivation of interfering components, and/or the addition of reagents. The subject and control sample may be derived from the same organism but may also be derived from different organisms/individuals. The subject sample may comprise tissue cultures or cell cultures. The subject and/or control sample may comprise the same kind of cell(s) and/or tissue(s).

Any cell type, tissue, or bodily fluid may be utilized to obtain a sample. Such cell types, tissues, and fluid may include sections of tissues such as biopsy and autopsy samples, frozen sections taken for histologic purposes, blood, plasma, serum, sputum, stool, tears, mucus, saliva, hair, and skin. Cell types and tissues may also include gastrointestinal cells or fluid, inflammatory tissue, premalignant adenomas, colorectal cancer, lymph fluid, ascetic fluid, gynecological fluid, urine, peritoneal fluid, cerebrospinal fluid, a fluid collected by vaginal rinsing, or a fluid collected by vaginal flushing. A tissue or cell type may be provided by removing a sample of cells from an organism, but can also be accomplished by using previously isolated cells (e.g., isolated by another person, at another time, and/or for another purpose). Archival tissues, such as those having treatment or outcome history, may also be used. The tissue may be an ovarian cancer tissue, a breast cancer tissue, a prostate cancer tissue, a lung cancer tissue, a gastric cancer tissue, a small intestine cancer tissue, and/or an inflamed tissue. The sample may be frozen, formalin-fixed, and/or paraffin-embedded. Nucleic acid purification may not be necessary.

(2) Subject

The subject may be a mammal. The mammal may be a human. The human may be healthy. The human may not exhibit symptoms of an illness. The human may be ill. The illness may be symptomatic of a disease. The illness may be elevated fever, high body temperature, low body temperature, hair loss, hyperpigmentation, skin rash, painful skin rash, fragile thin skin, skin that bruises easily, acne, sun sensitivity, skin thickening, skin ulcers, dry eyes, blurred vision, optic neuritis, eye discomfort or pain, dry mouth, hoarseness, difficulty in swallowing, mouth and/or nose sores, fullness or pressure, choking sensation in throat, chronic fatigue, insomnia, pain or tenderness throughout body, joint stiffness, deformed joints, carpal-tunnel syndrome, Raynaud's phenomenon (extreme sensitivity to cold in the hands and feet), swelling in hands and feet, weight loss, weight gain, weight gain in upper body or abdomen, rounded or puffy face, lack of coordination or unsteady gait, increased thirst, low blood pressure, high blood pressure, increased urination, nausea, vomiting, diarrhea, cysts on ovaries, irregular menstrual periods, recurrent miscarriage, reduced sex drive, decreased fertility, unexplained anemia, high cholesterol, delayed growth, high blood sugar, low blood sugar, and/or an increase in snoring.

The subject may be diagnosed with having a disease. The subject may be diagnosed as having a predisposition to develop a disease. The subject may be genetically predisposed to develop a disease. The disease may be cancer. The cancer may be brain, breast, skin, stomach, prostate, lung, and/or ovarian cancer. The ovarian cancer may be epithelial ovarian cancer (EOC).

(3) Vector

A vector may be used to express the PTB target gene, PTB target minigene, and/or the candidate modulator compound. The vector may be an expression vector. The expression vector may comprise one or more control sequences capable of enhancing, increasing, attenuating, suppressing, or inhibiting, the expression of the PTB target gene, PTB target minigene, and/or the candidate modulator compound. Control sequences that are suitable for expression in prokaryotes, for example, include a promoter sequence, an operator sequence, and a ribosome binding site. Control sequences for expression in eukaryotic cells may include a promoter, an enhancer, and a transcription termination sequence (i.e. a polyadenylation signal).

The expression vector may include other sequences. Expression vectors may comprise inducible or cell-type-specific promoters, enhancers or repressors, introns, polyadenylation signals, selectable markers, polylinkers, site-specific recombination sequences, and other features to improve functionality, convenience of use, and control over mRNA and/or protein expression levels. A signal sequence may direct the secretion of a polypeptide fused thereto from a cell expressing the protein. In the expression vector, nucleic acid encoding a signal sequence may be linked to a polypeptide coding sequence so as to preserve the reading frame of the polypeptide coding sequence.

The vector may be a plasmid, a phage, and/or a virus. The vector may be modified. The vector may be a lentiviral vector.

e. Control

The control may be PTB-expression or activity associated in a second cell. The control second cell may comprise PTB and the reporter system. The control second cell may be contacted with a modulator compound known to induce or enhance or suppress or inhibit or inhibit completely PTB-expression or activity.

The level(s) of expression and/or activity of PTB in a cell in contact with, or formerly in contact with, may be compared to the level(s) of expression and/or activity of PTB in the control second cell.

The control may be another PTB target gene or minigene. For example, the PTB target gene or minigene may be a second, third, fourth, fifth, or sixth or more target gene. The other PTB target gene or minigene may comprise the PTB target gene operably linked to a reporter sequence. The level(s) of reporter output may be indicative of PTB activity or lack thereof.

The control may be a cell treated with DMSO, which may serve as a negative control. The control may be a cell that expresses doxycycline-induced PTBsiRNA, which may serve as a positive control.

f. Recovery of Modulator Compound

Methods for recovery of the candidate modulator compound identified as modulating PTB expression and/or activity may vary depending on the expression system employed. A compound including a signal sequence may be recovered from the culture medium or the periplasm. The compound may be expressed intracellularly and recovered from the culture medium.

The expressed modulator compound, or candidate modulator compound, may be purified from culture medium or a cell lysate by any method capable of separating the compound from one or more components of the host cell or culture medium. The compound may be separated from host cell and/or culture medium components that would interfere with the intended use of the compound. As a first step, the culture medium or cell lysate may be centrifuged or filtered to remove cellular debris. The supernatant may then be concentrated or diluted to a desired volume or diafiltered into a suitable buffer to condition the preparation for further purification. The compound may then be further purified. The compound may be purified using an affinity column containing the cognate binding partner of a binding member of the compound. A compound fused with GFP, hemaglutinin, or FLAG epitope tags or with hexahistidine or similar metal affinity tags may be purified by fractionation on an affinity column.

Compounds identified by the herein described method may be further evaluated, detected, cloned, sequenced, and the like, either in solution or after binding to a solid support. For recovery of an expressed candidate compound, the host cell may be cultured under conditions suitable for cell growth and expression and the expressed compound recovered from a cell lysate or, if the candidate compounds are secreted, from the culture medium. The nutrients and growth factors are, in many cases, well known or may be readily determined empirically by those skilled in the art. Suitable culture conditions for mammalian host cells may be described in “Mammalian Cell Culture” (Mather ed., Plenum Press 1984) and in Barnes and Sato (Cell, 22:649 (1980)).

### 3. METHOD OF TREATING DISEASE

Also provided herein is a method for treating a subject diagnosed with, or predisposed to, a disease. The method may comprise administering the PTB modulator compound identified by the method described herein to a subject in need thereof. The subject may be a mammal. The mammal may be a human.

The subject may be diagnosed with having a disease. The subject may be diagnosed as having a predisposition to develop a disease. The subject may be genetically predisposed to develop a disease. The disease may be cancer. The cancer may be brain, breast, skin, stomach, prostate, lung, and/or ovarian cancer. The ovarian cancer may be epithelial ovarian cancer (EOC).

The present invention has multiple aspects, illustrated by the following non-limiting examples.

### EXAMPLES

### Example 1

### PTB Overexpression in Ovarian Tumors

Epithelial ovarian tumors overexpress PTB compared to their matched normal ovarian tissues. See PCT/U.S.07/07352, which is herein fully incorporated by reference. Based upon this finding, PTB expression in ovarian tumors with different malignancy and in invasive epithelial ovarian cancer (EOC) at different stages was evaluated. Two specialized tissue microarrays (TMAs), on (called Ovarian Disease Status TMA) containing benign ovarian tumors, borderline/low malignant potential (LMP) ovarian tumors as well as invasive EOC, and the other (called Ovarian Cancer Stage TMA) containing invasive EOC ranging from stage Ito stage IV disease, were evaluated by immunohistochemical staining for PTB. The result of average staining in the Ovarian Disease Status TMA is summarized in Table 3.

TABLE 3
Table 1. Average PTB staining in the Ovarian Disease Status TMA

| | Overall Expression † ‡ | | | |
|---|---|---|---|---|
| | All | | All | |
| Disease Status | Negative | Mixed | Positive | Total |
| Benign | 8 (47.1) | 6 (35.3) | 3 (17.6) | 17 |
| Borderline/LMP | 8 (16.3) | 12 (24.5) | 29 (59.2) | 49 |
| Invasive | 2 (3.0) | 8 (11.9) | 57 (85.1) | 67 |
| Total | 18 | 26 | 89 | 133 |

[symbol]

[symbol]

indicates data missing or illegible when filed

In Table 3, the average staining for each valid case (with a minimum of 2 satisfactory cores) is categorized into three groups: all negative (all evaluable cores were negative), all positive (all evaluable cores were positive), and mixed (at least one evaluable core negative and one evaluable core positive). Row percentage within each staining category is provided within parentheses. Statistical significance was evaluated using Fisher's exact test (2×2 tables) or its Mehta and Patel version (R×C tables). † Overall test: p=4.2×10-7 for benign vs. borderline/LMP vs. invasive tumors. ‡ Pair-wise comparisons: p=4.7×10-8 for benign vs. invasive, p=0.0069 for benign vs. borderline/LMP and p=0.0039 for borderline/LMP vs. invasive tumors.

As shown in the table, the percentage of cases stained all positive increases while the percentage of cases stained all negative or mixed decreases in the order of benign tumor, borderline/LMP tumor and invasive EOC. Approximately 85% EOC stained all positive for PTB, whereas a great majority of benign ovarian tumors stained all negative or mixed with only 17.6% stained all positive. The percentages of borderline/LMP ovarian tumors that stained all positive, all negative, or mixed fell between those of benign and invasive tumors. Statistical analyses indicated that the differences in PTB staining amoung benign, borderline?LMP and invasive ovarian tumors were significant in both overall comparison and all pair-wise comparisons. Analysis focusing on one subtype, i.e. mucinous tumors, generated the same results as above. In contrast, staining of Ovarian Cancer Stage TMA showed that great majority of cases were stained all positive and none stained all negative for PTB and there were no significant differences in average staining or frequency of positive cancer cell between any tumor stages. PTB expression may be associated with malignancy of ovarian tumors but not with stage of EOC.

### Example 2

### Immortalization of Ovarian Epithelial Cells Increases the Expression of PTB

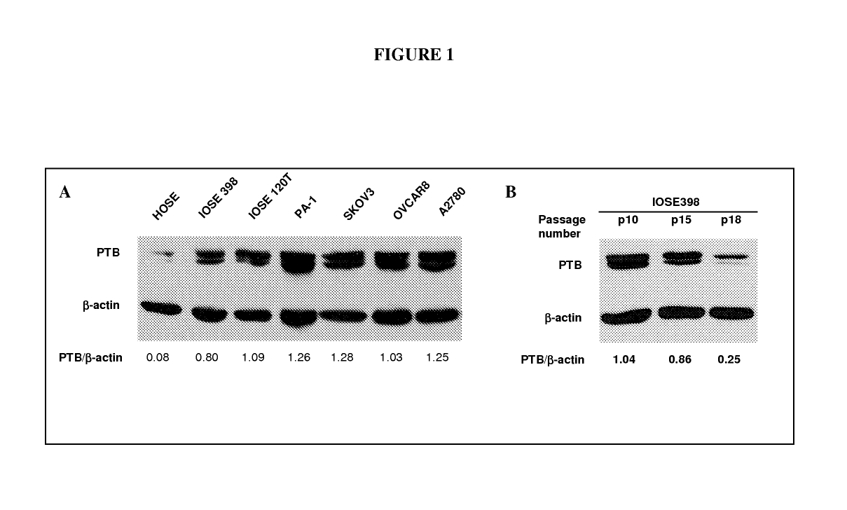

Expression of PTB was examined via western blot in normal human ovarian surface epithelia (HOSE), life-extended HOSE (105E398, HOSE transduced by SV40 T-antigen), truly immortalized HOSE (IOSE120T, HOSE sequentially transduced by SV40 T-antigen and hTERT) and ovarian epithelial tumor cell lines PA-1, SKOV3, OVCAR8 and A2780. As shown in FIG. 1A, the expression of PTB is substantially overexpressed in life-extended IOSE398 cells and maintained at high levels in IOSE120T and ovarian tumor cell lines, compared to normal HOSE cells. Further, we compared the PTB levels at different passages of IOSE398 cells, which senesce at around passage 20. As can be seen in FIG. 1B, PTB levels were gradually reduced when cells were approaching senescence. The up-regulation of PTB may be an early event in the neoplastic transformation of ovarian epithelial cells and may be required for cell growth. FIG. 1A shows immunoblotting analysis of PTB expression in cell lines. FIG. 1B shows PTB expression in IOSE398 cells at different passages. Multiple PTB bands are different splice variants of PTB. The expression levels of PTB are quantified as a ratio of PTB to β-actin expression.

### Example 3

### Knockdown of PTB Suppresses Ovarian Tumor Cell Growth Both In Vitro and In Vivo

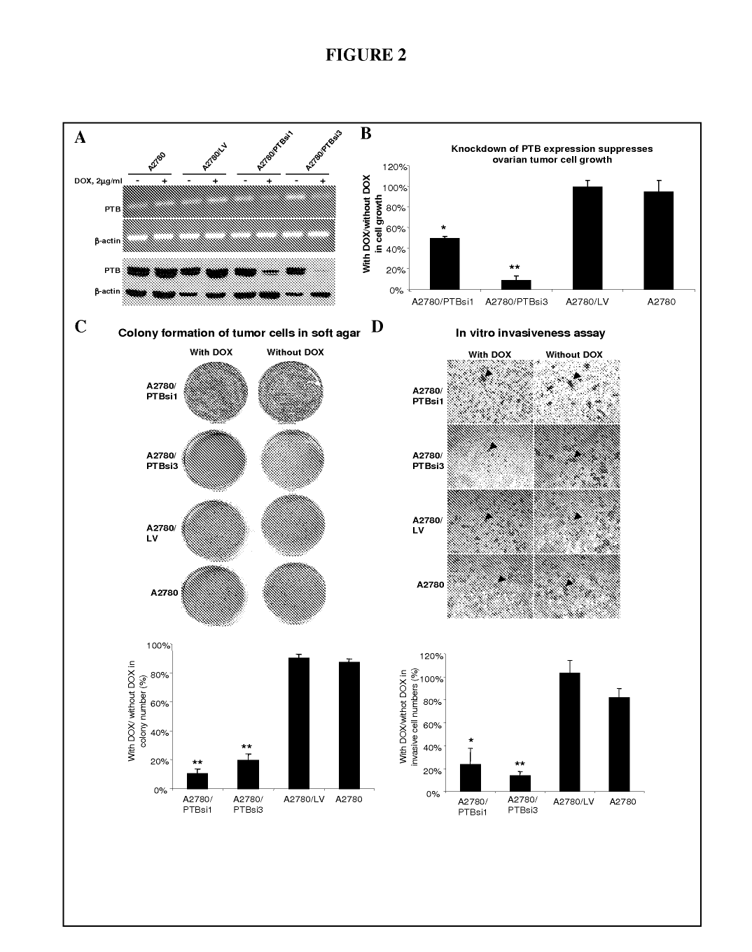

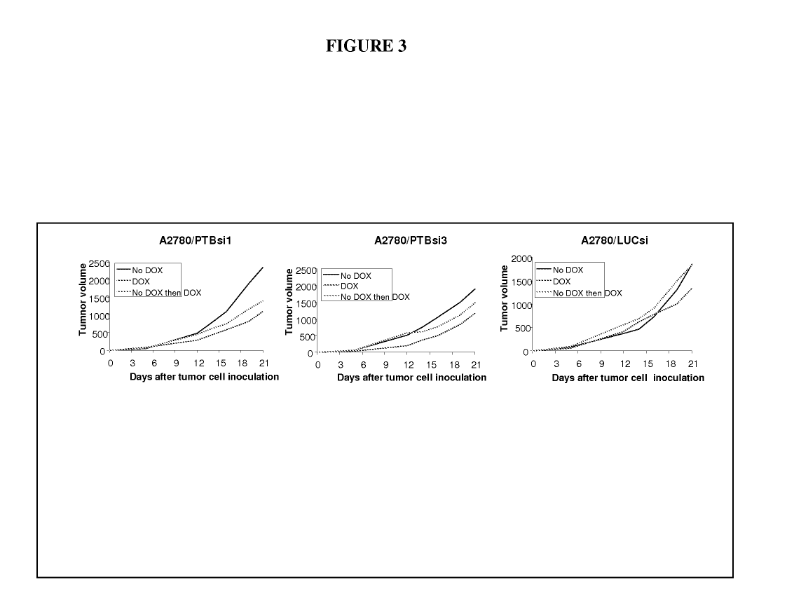

siRNA technology was .used to knock down the expression of PTB in tumor cells and to examine the effects of such manipulations on cell growth and malignant properties. Three siRNA (PTBsi1, PTBsi2, and PTBsi3) sequences targeting different regions of PTB mRNA were used. A siRNA can be generated as described in PCT/US2007/007352, which is herein fully incorporated by reference. Each of PTBsi1, PTBsi2, and PTBsi3 may be generated in a cell from a shRNA, which is formed after transcription of its coding sequence. The sequences of three pairs of oligonucleotides encoding for PTB shRNA1, shRNA2, and shRNA3 are shown in Table 3. The annealing of two oligonucleotides generates a DNA fragment with protruding ends compatible with Hind III and Bgl II restriction enzyme sites respectively. The coding sequences for each siRNA were cloned individually into a lentiviral vector downstream of H1 promoter and tetO element and thus the expression of these siRNAs is under control of doxycycline (DOX) (i.e. induced by DOX). We established several stable sublines carrying these expression cassettes. FIG. 2A shows the knockdown of PTB by DOX-induced siRNAs at both mRNA and protein levels; FIGS. 2B, 2C and 2D show the suppression of ovarian tumor cell growth, colony formation in soft agar and invasiveness by PTB knockdown. We have also examined whether knockdown of PTB could influence ovarian tumor cell growth in xenograft mouse model. Athymic nu/nu mice were inoculated A2780 sublines expressing DOX-induced PTB siRNA (A2780/PTBsi1 and A2780/PTBsi3) or luciferase siRNA (A2780/LUCsi) subcutaneously and then treated in three different ways: No DOX, with DOX or No DOX for 12 days and then with DOX. FIG. 3 shows the results of this in vivo experiment. It can be seen that tumors originated from sublines A2780/PTBsi1 and A2780/PTBsi3 grew slower in mice administered DOX either from the beginning of inoculation or 12 days after inoculation. Knockdown of PTB may suppress tumor growth in vivo. With respect to FIG. 2C, the upper part of the figure shows colonies in soft agar and the lower part shows average ratios (in percentage) of colony numbers formed with DOX vs. without DOX (n=3). With respect to FIG. 2D, the upper part shows invasive cells under microscope (40×) of one experiment and lower part shows average ratios (in percentage) of invasive cells grown with DOX vs without DOX (n=3). Invasive cells were counted under microscope with high magnification (150×). Arrows indicate the invasive cells. * and ** indicate P<0.05 and P<0.01, respectively, when compared to either control cell line. Error bars represent the standard error (SE). With respect to FIG. 3, Athymic nu/nu mice were injected subcutaneously at both flanks 5×10⁶ tumor cells in 0.1 ml PBS. Every mouse was inoculated two different subline cells, one at a flank, to maximize the randomness of distribution of sublines in mice. For each subline, there were 30 injections. After inoculation, mice were treated in three different ways: one group was given drinking water supplemented with 5% sucrose, another group was given drinking water supplemented with 5% sucrose and 2 mg/ml DOX, and the third group was first given drinking water supplemented with 5% sucrose for 12 days and then switched to drinking water supplemented with 5% sucrose and 2 mg/ml DOX for the rest of the experiment. Tumor sizes were measured in long and short dimensions every three or four days. Tumor size was calculated with formula axb2/2, where a=long dimension and b=short dimension. Mice were sacrificed on day 21 and day 23 because many tumors reached pre-defined tumor size limit.

TABLE 4

| shRNA No. and | |
|---|---|
| Oligonucleotide | |
| Sequences | SEQ ID NO. |
| shRNA1: | SEQ ID NO: 1 |
| 5′ GATCCCCAGGTGACAGC | |
| CGAAGTGCATTCAAGAGA | |
| TGCACTTCGGCTGTCACCT | |
| TTTTTGGAAA 3′ | |
| and | |
| 5′ AGCTTTTCCAAAAATGCA | SEQ ID NO: 2 |
| CTTCGGCTGTCACCTTCTCT | |
| TGAAAGGTGACAGCCGAAG | |
| TGCAGGG 3′ | |
| shRNA2: | SEQ ID NO: 3 |
| 5′ GATCCCCAACTTCCATCAT | |
| TCCAGAGAATTCAAGAGATT | |
| CTCTGGAATGATGGAAGTTT | |
| TTGGAAA 3′ | |
| and | |
| 5′ AGCTTTTCCAAAAATTCT | SEQ ID NO: 4 |
| CTGGAATGATGGAAGTCTC | |
| TTGAAAACTTCCATCATTCC | |
| AGAGAAGGG 3′ | |
| shRNA3: | SEQ ID NO: 5 |
| 5′ GATCCCTGACAAGAGCCG | |
| TGACTACTTCAAGAGAGTAG | |
| TCACGGCTCTTGTCATTTTTG | |
| GAAA 3′ | |
| and | |
| 5′ AGCTTTTCCAAAAATGACAA | SEQ ID NO: 6 |
| GAGCCGTGACTACTCTCTTGAA | |
| GTAGTCACGGCTCTTGTCAGGG 3′ | |

### Example 4

### Knockdown of PTB Alters Splicing Pattern and Expression Profile in the Cell

Microarray analyses of genome-wide splicing patterns and gene expression profiling were performed in A2780/PTBsi3 cells with or without PTB knockdown. Gene expression profiling was assessed using Affymetrix HG-U133 plus 2 oligonucleotide arrays, and the splicing pattern was assessed by Jivan Biologics' splicing-sensitive microarrays. The former analysis was done three times and the latter was done twice with total RNAs isolated from separate experiments. We also examined the gene expression profile once in the control subline, A2780/LUCsi, grown with or without DOX.

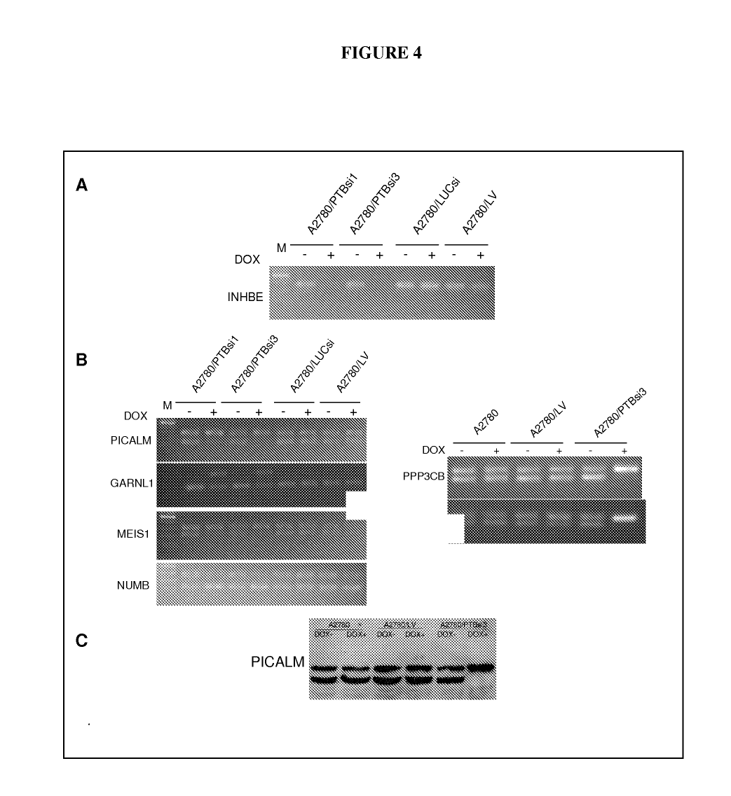

DOX treatment caused very few changes in gene expression of the controls. With signal intensity of 200 as a cutoff, only 7 genes were found to be changed more than 2-fold, among which 3 have annotation data available and other 4 do not. However, in A2780/PTBsi3 cells treated with DOX (i.e. PTB knockdown), 52 genes were found consistently changed more than 2-fold in all three separate experiments. As an indication of the reliability of the assay, PTB was among these genes and consistently downregulated about 4-fold in all three experiments. Between these genes and those identified in the control cell line, there was one in common, which was dramatically increased in all DOX treated cells, indicating it was not induced by PTB knockdown. In total, we found 50 genes whose expression was regulated by PTB. Among these genes, 27 were downregulated and 23 were upregulated. We have validated these changes in a few of these genes (INHBE, CDC42, PTBP2 and PKP4) by real-time PCR or conventional RT-PCR. As an example, FIG. 4A shows the result of validation of INHBE expression.

From the splicing-sensitive microarray analysis, we identified 317 genes whose splicing patterns were consistently altered more than 2-fold after PTB knockdown in two separate experiments. Table 4 shows the summary of these alterations. The results indicate that PTB's role in alternative splicing is much broader than previously thought as a splicing repressor enhancing exon skipping. It actually participates in all kinds of splicing events including exon skipping, alternate use of exons, exon trimming and intron retention. It is worth noting that many genes underwent changes in two or more splicing events after PTB knockdown. Therefore, the total number of genes is less than the sum of individual numbers in the Table. Seventeen genes with altered expression levels by Affymetrix microarray analyses were also found altered in their splicing patterns, so it is very likely that the changes in mRNA levels of some genes may reflect switches in splicing patterns rather than transcription. We have picked seven differentially expressed splice variants found by the microarray analysis in order to further examine their expression using conventional RT-PCR and have validated six, as shown in FIG. 4B. We also validated PICALM at protein level (FIG. 4C). These splice variants are all new targets of PTB regulation of alternative splicing not previously reported.

TABLE 5
Table 2. Summary of alternative splicing influenced by PTB

| | Genes with 2 or more fold change in two |
|---|---|
| Splicing events | experiments |
| ExonSkip | 190 |
| Alt1stExon | 62 |
| AltLastExon | 48 |
| Alt3Donor | 16 |
| Alt5Acceptor | 14 |
| IntronRetention | 21 |
| Total unique genes | 317 |

### Example 5

### A Reporter System for Detection of PTB Activity in the Cell

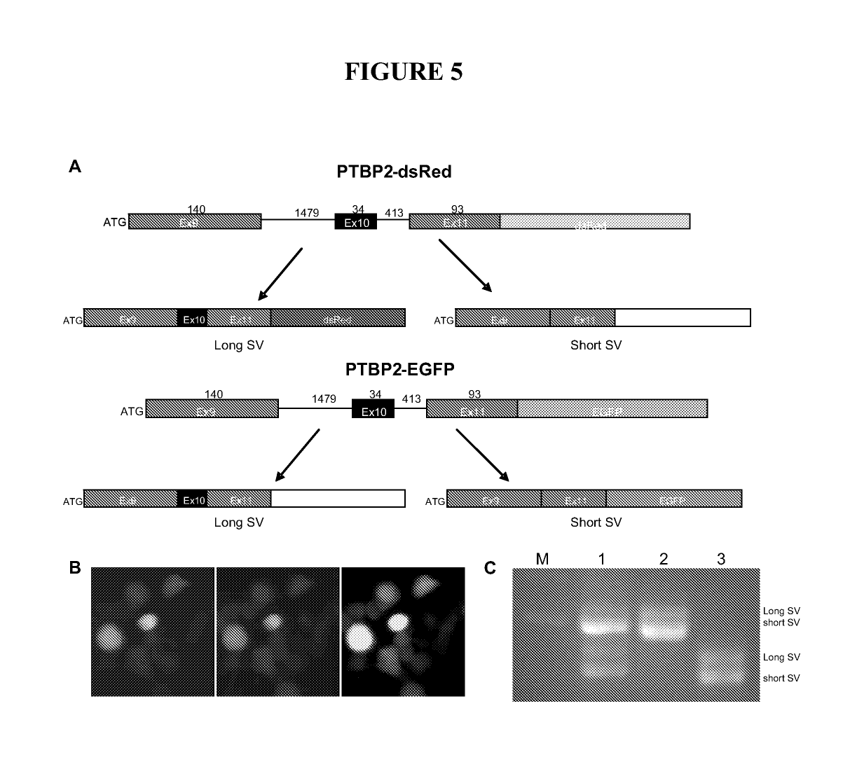

The results presented above (see FIGS. 2 and 3) suggest that PTB may be a good therapeutic target for ovarian cancer. In this application, we intend to test this idea by high throughput screening for small molecules that inhibit PTB activity in the cell. In order to do this, we have begun to develop a reporter system to monitor the activity of PTB. As described earlier, a major function of PTB is to regulate alternative splicing. Accordingly, our reporter system monitors PTB activity by detecting alternative splicing regulated by PTB. As shown in FIG. 4B, splicing of PTBP2 exon 10 was controlled by PTB. Knockdown of PTB almost completely suppressed skipping of exon 10, resulting in a single transcript with exon 10 included. Our reporter system makes use of this characteristic of PTB. It is a minigene construct containing the genomic sequence spanning exon 9 to exon 11 of PTBP2, which is immediately upstream of the coding sequence of dsRed or EGFP. FIG. 5A shows the schematic diagram of the construct. The genomic sequence was obtained by PCR from A2780 cell genomic DNA with a start codon, ATG, added at 5′ end of the exon 9. Exon 10 is composed of 34 nucleotides and its skipping will cause a shift of the downstream reading frame. We engineered one construct (called PTBP2-dsRed) so that the downstream dsRed coding sequence will be in-frame when all three PTBP2 exons are spliced together and out of frame when exon is 10 skipped. In the other construct (called PTBP2-EGFP), we engineered it so that the downstream EGFP is out of frame when all three exons are spliced together but will be in frame when exon 10 is skipped. Therefore, the former construct can report the existence of the big variant with three exons included and the latter construct can report the existence of the small variant with exon 10 skipped. When these two constructs are introduced into cells expressing high levels of PTB, such as 293T cells and A2780 cells, we will see both dsRed and EGFP expressed because both PTBP2 splice variants are generated in such cells. FIG. 5B shows the experimental results of co-transfection of these two constructs into 293T cells. RT-PCR indicated that the PTBP2 exons in the constructs were spliced together to form two distinct variants as expected (FIG. 5C). These results confirm the feasibility of using these constructs to detect the alternative splicing of PTBP2 exon 10. These constructs may be used to monitor the levels of PTB in the cell. We expect to see an increase of red fluorescence and a decrease of green fluorescence when PTB is knocked down or its activity is inhibited, because under this condition the expression of the big variant will increase while the expression of the small variant will decrease (see FIG. 4).

As shown above, we have constructed two plasmids to carry the PTBP2 minigene spanning exon 9 to exon 11 and that the minigene can be correctly processed into two distinct splice variants in the cell to tell the splicing status of exon 10. Because the splicing of the exon 10 is controlled by PTB (see FIG. 4), PTB's expression or activity may be monitored through the detection of splicing of PTBP2 exon 10. To test the capability of these two constructs in this regard, we will first test whether they can monitor the down-regulation of PTB expression by siRNA. If confirmed, we will then optimize this reporter system for later high throughput screening. With respect to FIG. 5A, a schematic diagram of the constructs to report the alternative splicing of PTBP2 exon 10 is shown. The rectangular bars represent exons and the lines represent the introns. The numbers above them are the length of corresponding exons and introns. FIG. 5B shows co-transfection of 293T cell with the two constructs. Left, red fluorescence; middle, green fluorescence; right, merged image. FIG. 5C shows RT-PCR of the SVs of PTBP2-dsRed and PTBP2-EGFP. The forward primer is on exon 9 and the reverse primer is on dsRed or EGFP. M: marker; 1: co-transfection with PTBP2-dsRed and PTBP2-EGFP; 2: transfection with PTBP2-dsRed; 3: transfection with PTBP2-EGFP.

### Example 6

### Construction of Lentiviral Vectors to Carry PTBP2-dsRed and PTBP2-EGFP and Preparation of Corresponding Lentiviruses

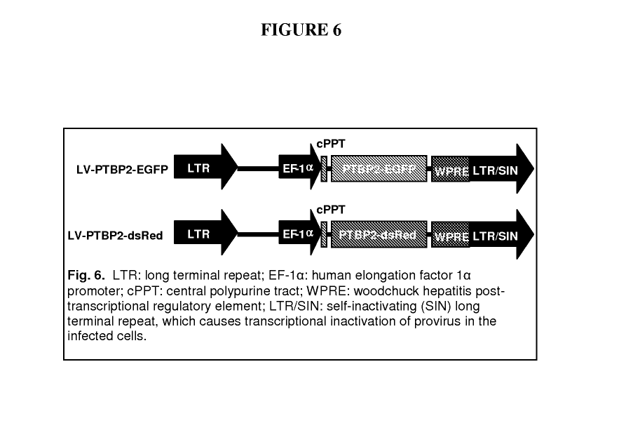

The backbone of our current constructs is from plasmid pEGFP-N1 (Clontech, Mountain View, Calif.). We cloned the PTBP2 genomic DNA into this vector at the sites EcoRI and BamHI. Because of limitations with transient plasmid transfection such as low efficiency and short retention period, it is more convenient to use lentiviruses to deliver the minigene constructs into the cell and express them. We obtained several lentiviral vectors as well as packaging plasmids from Dr. Didier Trono (University of Geneva, Switzerland). The specific lentiviral vector that will be used to carry and express PTBP2-dsRed and PTBP2-EGFP is LV-tTR/KRAB. We will replace the tTR/KRAB in this vector with PTBP2-EGFP or PTBP2-dsRed to generate LV-PTBP2-EGFP and LV-PTBP2-dsRed, as shown in FIG. 6. Regular cloning techniques will be employed to accomplish this. Once these lentiviral vectors are prepared, we will make lentiviruses in HEK293T cells using the second generation packaging system, which includes pMD2G (expressing vesicular stomatitis virus G envelope protein (VSV-G)) and psPAX2 (expressing the components necessary for packaging). We routinely generate high titers of lentiviruses (>10⁷) without concentration.

### Example 7

### Modification of Lentiviral Vectors Carrying the Expression Cassette for DOX-Inducible PTB siRNA

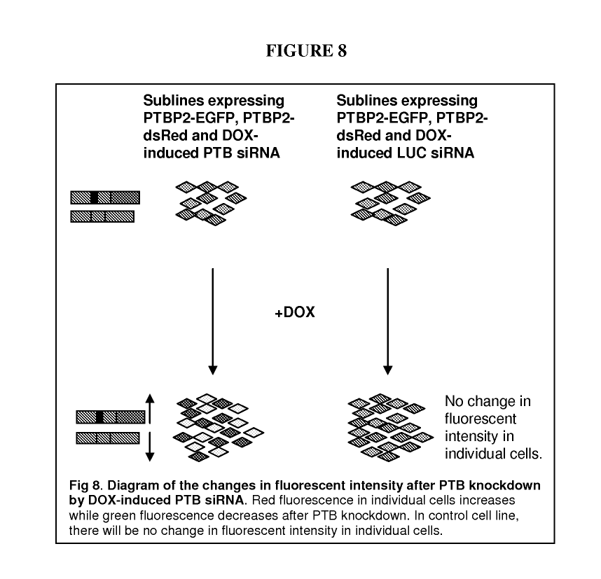

The vector-based DOX-inducible PTB siRNA will be used to knockdown PTB expression in the cell to test the above reporter system. However, the lentiviral vectors we made previously to express DOX-inducible PTB siRNA or control siRNA also express EGFP and thus are not suitable for this purpose. Therefore, we will replace the coding sequence for EGFP from these vectors with the puromycin resistant gene by regular cloning techniques. The resulting lentiviral vector is depicted in FIG. 8. Cells transduced by this vector only will express short hairpin RNA (shRNA) constitutively. If the cells are also transduced by LV-tTR/KRAB, which harbors the expression cassette of a fusion protein of tet repressor and KRAB, then the expression of shRNA become DOX-inducible.

### Example 8

### Establishment of Sublines to Express PTBP2-EGFP and PTBP2-dsRed

In this application, our focus is on ovarian cancer. Therefore, we will use a panel of ovarian cancer cell lines to test the reporter system. In our previous work, A2780 cells were used to study the effects of PTB knockdown. Hence, we will start with this ovarian cancer cell line. Co-infection of A2780 cells with lentiviruses LV-PTBP2-EGFP and LV-PTBP2-dsRed will be done in 12-well plate. Infected cell will then be seeded into 10 cm-dishes at very low density (300 to 400 cells per dish) to allow the formation of colonies. Cell colonies exhibiting both red and green fluorescence will be picked and expanded. Expression of long and short SVs of PTBP2-dsRed and PTBP2-EGFP in these cell clones will be further confirmed by RT-PCR as we did in FIG. 5C. Other ovarian cancer cell lines to be tested are OVCAR3, OVCAR4, OVCAR5, OVCAR8, IGR-OV1 and SKOV3. They belong to NCI 60 human cancer cell lines used for in vitro drug screening and are different in their phenotypes such as responses to hormones or chemotherapeutic agents. The ideal cell lines for high throughput screening, to be determined here, will be those expressing low intensity of red fluorescence and high intensity of green fluorescence. We will use optical filter sets with excitation/emission wave lengths (nm) 558/583 and 488/507 to detect dsRed and EGFP fluorescent intensity, respectively, in all experiments in this application.

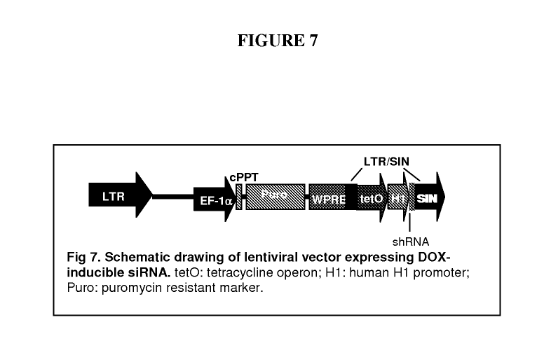

There are two ways to knock down PTB expression by siRNA in the cells. One is to express PTB siRNA constitutively and the other is to express DOX-induced PTB siRNA. For the purpose of HTS, it is better to test the reporter system with the second way because it resembles the drug treatment in HTS the best. Therefore, we will establish secondary sublines to express DOX-inducible PTB siRNA on the basis of the above sublines. Cells of the above sublines will be co-infected by lentiviruses carrying PTB siRNA or luciferase (LUC) siRNA (see FIG. 7) and lentiviruses carrying tTR/KRAB, which binds to the tetO to inhibit the expression of downstream gene. Cell colonies formed after puromycin selection will be picked and expanded. Afterwards, these cells will be examined with Western Blot for the downregulation of PTB by DOX induction (2 μg/ml). Clones with greater than 75% PTB knockdown after DOX induction for 5 days will be further examined to determine the time-course and the dose-response relationship of DOX induction. The sublines displaying approximate linear time-course and dose-response on normal or semi-log graph in DOX-induced PTB knockdown in a 7 days' window and dose range of 10-4 to 2 μg/ml will be subject to further test described below.

### Example 9

### Reporter System Test

Once the above secondary sublines are established, we will test how well they can monitor the downregulation of PTB by siRNA. Initially, we will perform this test in 12-well plate. After we identify the best clones, we will test them in 96-well and 384-well plate for HTS (see below). Cells will be seeded into the wells and DOX will be added right after. The intensity of red and green fluorescence will be measured in a fluorescent plate reader at 24, 48, 72, 96, 120, 144 and 168 hours after DOX addition. The expected result is diagramed in FIG. 8. In the cells expressing DOX-inducible PTB siRNA, after DOX addition, red fluorescence will be intensified while green fluorescence diminished, because the long form PTBP2-dsRed is to be upregulated and short form PTBP2-EGFP downregulated by PTB knockdown, and thus the ratio of red fluorescence vs green fluorescence will increase. In contrast, in control cells, DOX addition will not change the intensity of red or green fluorescence and thus the ratio is suppose to stay the same. After the measurement of fluorescent intensity, cells will be harvested and PTB protein levels will be analyzed by immunoblotting. The correlations between PTB levels and intensities of red and green fluorescence will then be established. The ideal clones should display negative correlation between PTB levels and red fluorescent intensity (i.e. more PTB, low red fluorescence; less PTB, high red fluorescence) and positive correlation between PTB levels and green fluorescent intensity. Nonetheless, the clones displaying only negative correlation between PTB levels and red fluorescence should be also useful, because the reduction of the PTBP2-EGFP might be slow even though its production is decreased when PTB is knocked down. In this situation, the ratio will still increase.

### Example 10

### Adaptation and Optimization of the Reporter System for High Throughput Screening (HTS)

In order to apply the above reporter system to HTS, it is necessary to adapt it to 96-well or 384-well microtiter plate format so that it can then be automated for large-scale HTS. As described above, the assay with the reporter system consists of basic four steps: 1.) seeding of cells, 2.) addition of DOX (for positive control) or compounds (from libraries) or DMSO alone (for negative control), 3.) incubation of cells and 4.) measurement of fluorescent intensity. Therefore, adaptation will revolve around the optimization of each of these steps. Specifically, we will perform experiments to address issues about solvent tolerance, optimal cell seeding density, plate uniformity and reproducibility. The primary statistical parameters we will use to judge the results and quality of the assay development are signal-to-background ratio and the Z′ factor. We describe these parameters in more detail in the data analysis section below.

For 96-well plates, we plan to test four seeding cell densities: 250, 500, 1000 and 2000 cells in 100 μl medium per well. For 384-well plates, the seeding cell densities will be 100, 200, 400 and 800 cells in 30 μl per well. Twenty-four h after seeding, DOX in 1×PBS or 1×PBS only will be added to the cultures at minimum concentration that gives rise to the greatest knockdown of PTB (it is determined in D.1.5 above). The fluorescent intensity of dsRed and EGFP will be monitored daily until 7 days after DOX addition.

Because small molecular compounds in libraries are dissolved in DMSO, adding the compounds directly to the cultures will also introduce DMSO. Therefore, it is necessary to assess the influence of low concentration of DMSO on cell growth and signal detection. Since compound libraries are typically composed of solutions of 10 mM compound dissolved in 100% DMSO, the dilution range of the compound into the cell culture is usually between 200 to 1000 times, the final concentration of DMSO in cell culture is between 0.1% and 0.5%. Therefore, we will test DMSO tolerance at these two concentrations.

One of the crucial yet often overlooked components of an HTS facility is data analysis. We have invested a significant amount of time and effort in search of low cost, high return solutions for the facility that all users and collaborators can afford and utilize. We have written our own PerlScript routines for integrating our microplate reader output with our compound library data, and have integrated this data with HTS Benchware software (Tripos) for cluster analysis and hit evaluation. For follow-up assays, we are using the CambridgeSoft BioAsssay software suite for rapid 1050 evaluation.

The first step in assay optimization and HTS data analysis is to determine and monitor the Z- or Z′-factor of the assay being developed or implemented. This simple statistical parameter was introduced by Zhang et al. to access the quality and utility of any HTS assay. The general equation for the Z-factor is Z′=1 (3σ+control+3σ-control)/lμ+control−μ-controll, where σ is the standard deviation and μ is the mean of signals. This coefficient is reflective of both the dynamic range of the assay signals and the data variation associated with the measurements. Z-factors between 0.5-1.0 indicate an excellent assay with 1.0 being designated as a perfect assay.

For development and transition of the PTB inhibition assay, Z′ factors will be calculated from data obtained above. For each cell seeding density or each DMSO concentration, at least three tests will be performed and in each test, at least one 96-well or one 384-well plate will be used. The layout of DOX treatment and DMSO treatment in a 96-well plate is shown below in Table 6.

TABLE 6

| Row | A | B | C | D | E | F | G | H | I | J | K | L |
|---|---|---|---|---|---|---|---|---|---|---|---|---|
| 1 | DOX | DMSO | DOX | DMSO | DOX | DMSO | DOX | DMSO | DOX | DMSO | DOX | DMSO |
| 2 | DOX | DMSO | DOX | DMSO | DOX | DMSO | DOX | DMSO | DOX | DMSO | DOX | DMSO |
| 3 | DOX | DMSO | DOX | DMSO | DOX | DMSO | DOX | DMSO | DOX | DMSO | DOX | DMSO |
| 4 | DOX | DMSO | DOX | DMSO | DOX | DMSO | DOX | DMSO | DOX | DMSO | DOX | DMSO |
| 5 | DOX | DMSO | DOX | DMSO | DOX | DMSO | DOX | DMSO | DOX | DMSO | DOX | DMSO |
| 6 | DOX | DMSO | DOX | DMSO | DOX | DMSO | DOX | DMSO | DOX | DMSO | DOX | DMSO |
| 7 | DOX | DMSO | DOX | DMSO | DOX | DMSO | DOX | DMSO | DOX | DMSO | DOX | DMSO |
| 8 | DOX | DMSO | DOX | DMSO | DOX | DMSO | DOX | DMSO | DOX | DMSO | DOX | DMSO |

After measuring fluorescent intensities with appropriate filters, the means and standard deviations of the ratios of red fluorescent intensity/green fluorescence intensity in all DOX-treated wells and all DMSO-treated wells of a plate will be calculated, respectively. With these two statistics, Z′ factors can be calculated using above-mentioned formula.

The Z′-factors will be monitored continuously when transitioning the assay from the bench, using 96-well plates, to the automated Tecan robot platform that will also be performed first in 96-well plates and then transitioned to 384-well plates if possible. Once the assay is optimized for the maximum Z′-factor, the library will be screened in duplicate. Z′-factors are calculated for each plate during the screening process to ascertain whether or not any problems arise over time, e.g. over the course of hours or days. Each 384-well plate will have 320 compounds, 32 positive controls and 32 negative controls (no compound).

### Example 11

### A Reporter System for Detection of PTB Activity in the Cell

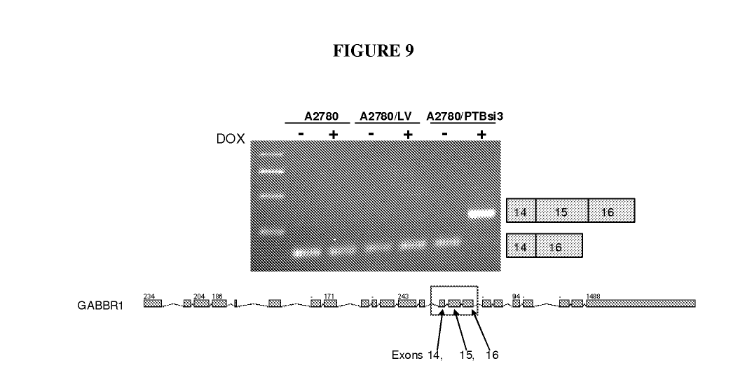

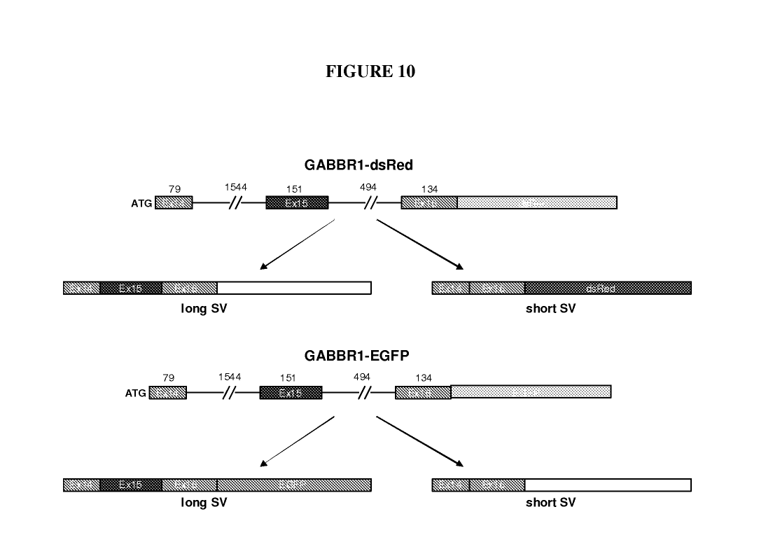

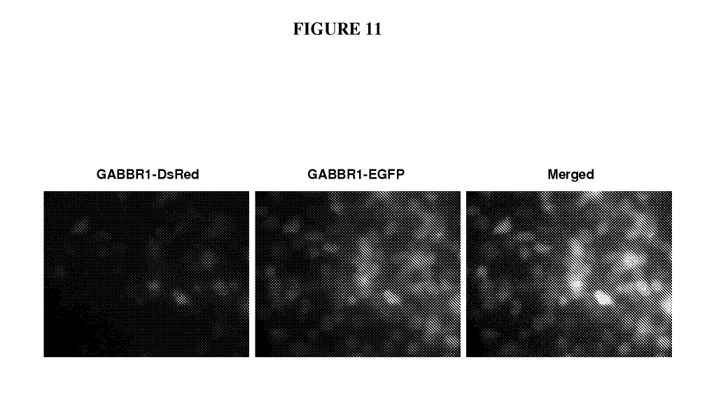

In this system, the minigene construct contains the genomic sequence spanning exon 14 to exon 16 of GABBR1 (gamma-aminobutyric acid (GABA) B receptor, 1) gene, which is immediately upstream of coding sequence of dsRed1 or EGFP. As shown in FIG. 9, the splicing of exon 15 of GABBR1 is regulated by PTB expression. In cells without PTB knockdown, the major splice variant of GABBR1 has exon 15 skipped while in cells with PTB knockdown, the major splice variant of GABBR1 has exon 15 included. FIG. 10 shows the schematic diagram of the construct. The genomic sequence spanning exon 14 to exon 16 of GABBR1 was amplified from human genomic DNA with the start codon ATG added to the 5′ end of exon 14. The amplified genomic fragment was then cloned into pDsRed-N1 and pEGFP-N1 vector between EcoRI and BamHI sites with DsRed or EGFP immediately downstream of GABBR1 genomic sequence. Both resultant expression vectors are expected to express long-form (with exon 15 included) and short-form (exon 15 skipped) GABBR1 splice variants (SVs). Without any sequence modification, DsRed and EGFP are out of reading frame in long-form SVs but in frame in short-form SVs. In order to detect the long-form SV of GABBR1, we cut the resultant expression vector derived from pEGFP-N1 with BamHI enzyme and then removed the overhang ends by mung bean nuclease followed by re-ligation of the plasmid. Such manipulation of the plasmid places the EGFP coding sequence in-frame when expressed with the long-form SV. The expression cassettes constructed above are called GABBR1-DsRed and GABBR1-EGFP, respectively. As shown in FIG. 11, after co-transfection of 293T cells with these vectors, we observed the expression of DsRed and EGFP in the cells, which indicates short-form and long-form SVs of GABBR1, respectively. Cells with high levels of PTB should exhibit more of the short-splice form of GABBR1 than the long form, as expressed in greater red fluorescence intensity, whereas in cells with PTB knocked down, we expect to see more long splice forms of GABBR1 than the short form, and will be expressed in more green fluorescence intensity.

We claim:

1. A method of screening for a modulator of PTB comprising:
(a) providing a cell comprising PTB and a reporter system, wherein the reporter system comprises a first PTB target gene operably linked to a reporter sequence;
(b) contacting the cell with a candidate modulator compound; and
(c) measuring the level of expression of the PTB target gene;
wherein a modulator of PTB is identified by a change in expression compared to a control.

2. The method of claim 1, wherein first PTB target gene encodes a protein that is selected from the group consisting of c-src, α-actinin, FGF-R2, calcitonin/CGRP, GABA_{A}γ2, α-tropomyosin, PTB1, PTB2, PTB4, GABBR1, INHBE, PICALM, GARNL1, MEIS1, NUMB, PPP3CB, and PTBP2.

3. The method of claim 1, wherein the first PTB target gene is a first minigene, wherein the minigene encodes a fragment of a protein selected from the group consisting of c-src, α-actinin, FGF-R2, calcitonin/CGRP, GABA_{A}γ2, α-tropomyosin, PTB1, PTB2, PTB4, GABBR1, INHBE, PICALM, GARNL1, MEIS1, NUMB, PPP3CB, and PTBP2.

4. The method of claim 3, wherein the first minigene comprises a fragment selected from the group consisting of exon 9 to exon 11 of PTBP2 and exon 14 to exon 16 of GABBR1.

5. The method of claim 4, wherein the first minigene further comprises exon 11 of PTBP2 operably linked to a first reporter.

6. The method of claim 4, wherein the first minigene further comprises exon 16 of GABBR1 operably linked to a first reporter.

7. The method of claim 1, wherein the reporter system further comprises a second PTB target gene.

8. The method of claim 7, wherein second PTB target gene encodes a protein that is selected from the group consisting of c-src, α-actinin, FGF-R2, calcitonin/CGRP, GABA_{A}γ2, α-tropomyosin, PTB1, PTB2, PTB4, GABBR1, INHBE, PICALM, GARNL1, MEIS1, NUMB, PPP3CB, PTBP2, and or a fragment thereof.

9. The method of claim 7, wherein the second PTB target gene is a second minigene, wherein the second minigene encodes a fragment of a protein selected from the group consisting of c-src, α-actinin, FGF-R2, calcitonin/CGRP, GABA_{A}γ2, α-tropomyosin, PTB1, PTB2, PTB4, GABBR1, INHBE, PICALM, GARNL1, MEIS1, NUMB, PPP3CB, and PTBP2.

10. The method of claim 9, wherein the second minigene comprises a fragment selected from the group consisting of exon 9 to exon 11 of PTBP2 and exon 14 to exon 16 of GABBR1.

11. The method of claim 10, wherein the second minigene further comprises exon 11 of PTBP2 operably linked to a second reporter.

12. The method of claim 10, wherein the second minigene further comprises exon 16 of GABBR1 operably linked to a second reporter.

13. The method of claim 5 or claim 11, wherein exon 10 of PTBP2 is skipped from the transcript of the first PTB target and the first reporter is not functionally expressed, thereby indicating that the candidate modulator does not inhibit PTB activity.

14. The method of claim 5 or claim 12, wherein exon 15 of GABBR1 is skipped from the transcript of the first PTB target and the first reporter is not functionally expressed, thereby indicating that the candidate modulator does not inhibit PTB activity.

15. The method of claim 11, wherein exon 10 of PTBP2 is skipped from the transcript of the second PTB target and the second reporter is functionally expressed, thereby indicating that the candidate modulator does not inhibit PTB activity.

16. The method of claim 12, wherein exon 15 of GABBR1 is skipped from the transcript of the second PTB target and the second reporter is functionally expressed, thereby indicating that the candidate modulator does not inhibit PTB activity.

17. The method of claim 5 or claim 11, wherein exon 10 of PTBP2 is included in the transcript of the first PTB target and the first reporter is functionally expressed thereby indicating that the candidate modulator inhibits PTB activity.

18. The method of claim 5 or claim 12, wherein exon 15 of GABBR1 is included in the transcript of the first PTB target and the first reporter is functionally expressed thereby indicating that the candidate modulator inhibits PTB activity.

19. The method of claim 11, wherein exon 11 of PTBP2 is included in the transcript of the second PTB target and the second reporter is not functionally expressed thereby indicating that the candidate modulator inhibits PTB activity.

20. The method of claim 12, wherein exon 15 of GABBR1 is included in the transcript of the second PTB target and the second reporter is not functionally expressed thereby indicating that the candidate modulator inhibits PTB activity.

21. The method of claim 17, wherein the expression of the first reporter is increased compared to a positive control.

22. The method of claim 15, wherein the expression of the second reporter is increased compared to a negative control.

23. The method of claim 16, wherein the expression of the second reporter is increased compared to a negative control.

24. The method of claim 1, wherein the level of expression is measured by level of target PTB gene encoded mRNA.

25. The method of claim 24, wherein the level of target PTB gene encoded mRNA is measure by RT-PCR.

26. The method of claim 5 or claim 12, wherein the level of expression is measured by reporter output.

27. The method of claim 26, wherein the reporter output is fluorescence.

28. The method of claim 1, wherein the cell is mammalian.

29. The method of claim 1, wherein the cell is non-mammalian.

30. The method of claim 1, wherein the PTB is endogenously expressed.

31. The method of claim 1, wherein the PTB is expressed from heterologous nucleic acid.

32. The method of claim 1, wherein the candidate modulator compound is from a library of compounds.

33. The method of claim 32, wherein the library of compounds is selected from the group consisting of a peptide library, a natural products library, a cDNA library, a combinatorial library, a drug library, an oligosaccharide library, and a phage display library.

34. The method of claim 1, wherein the candidate compound is endogenously expressed.

35. The method of claim 1, wherein the control is an expression level of the PTB-target gene of claim 1 that is associated with a modulator compound known to suppress or enhance PTB expression or activity.

36. The method of claim 1, wherein the screen is a high-throughput screen.

37. A method of treating a subject predisposed or diagnosed with a disease, comprising administering the compound identified in claim 1.

38. The method of claim 37, wherein the disease is cancer.

39. The method of claim 38, wherein the cancer is ovarian cancer.


SEQUENCE LISTING (6 sequences)

SEQ ID NO: 1 - DNA, 64 bp, Artificial Sequence
  Oligonucleotide.
gatccccaggtgacagccgaagtgcattcaagagatgcacttcggctgtcaccttttttggaaa

SEQ ID NO: 2 - DNA, 64 bp, Artificial
  Oligonucleotide.
agcttttccaaaaatgcacttcggctgtcaccttctcttgaaaggtgacagccgaagtgcaggg

SEQ ID NO: 3 - DNA, 66 bp, Artificial
  Oligonucleotide.
gatccccaacttccatcattccagagaattcaagagattctctggaatgatggaagtttttggaaa

SEQ ID NO: 4 - DNA, 66 bp, Artificial
  Oligonucleotide.
agcttttccaaaaattctctggaatgatggaagtctcttgaaaacttccatcattccagagaaggg

SEQ ID NO: 5 - DNA, 63 bp, Artificial
  Oligonucleotide.
gatccctgacaagagccgtgactacttcaagagagtagtcacggctcttgtcatttttggaaa

SEQ ID NO: 6 - DNA, 64 bp, Artificial
  Oligonucleotide.
agcttttccaaaaatgacaagagccgtgactactctcttgaagtagtcacggctcttgtcaggg